(19) United States
(12) Patent Application Publication
(10) Pub. No.: US 2025/0122465 A1
(43) Pub. Date: Apr. 17, 2025

(54) Compositions and Methods for Germinating Wild-Type or Super-Dormant Bacillus Strains

(71) Applicant: NCH Corporation, Irving, TX (US)

(72) Inventors: Edidiong Inyang, Arlington, TX (US); Haoqin Zhou, Euless, TX (US); Skylar White, Weatherford, TX (US); Charles Greenwald, Irving, TX (US)

(21) Appl. No.: 18/914,868

(22) Filed: Oct. 14, 2024

Related U.S. Application Data
(60) Provisional application No. 63/590,319, filed on Oct. 13, 2023.

(51) Int. Cl. C12N 1/20

(52) U.S. Cl. CPC C12N 1/20

(57) ABSTRACT

A germinant composition for activating one or more wild-type and/or super-dormant spores, particularly Bacillus spores, comprises L-isoleucine or L-valine or both. A germinant composition may also comprise a buffer, a source of potassium, an industrial preservative, and one or more other L-amino acids. Wild-type and/or super-dormant spores may be incubated with a germinant composition by heating to an incubation temperature of 26 to 60° C. for an incubation period of 30 minutes to 36 hours. Incubation may be a single step process without pre-activation or heat shock or a two-step process. A method of pre-activation comprises combining spores with a germinant composition and either bioprocessing or heat treating and then drying. An incubated bacteria solution preferably comprises at least 50% activated spores after an incubation period of 24 hours and more preferably after 1 hour.

### CROSS REFERENCE TO RELATED APPLICATION

This application claims the benefit of U.S. Provisional Application Ser. No. 63/590,319 filed on Oct. 13, 2023.

### BACKGROUND OF THE DISCLOSURE

### 1. Field of the Disclosure

The present disclosure relates to an activation media or nutrient-germinant composition for activating or germinating wild strains and/or super-dormant strains of bacteria, particularly super-dormant Bacillus species, and a method for activating or germinating such species.

### 2. Description of Related Art

Bacillus spore species exhibit remarkable resilience against various environmental factors, allowing them to remain dormant for extended periods. Nevertheless, these spores can be reactivated under specific conditions and with the availability of particular germinants, commencing the germination process. Spore germination is a multistep, causative process wherein spores effectively wake-up or are revived from a dormant state to a vegetative growth state. The first step is one by which spores are activated and are induced to germinate, typically by the presence of a nutrient germinant. Indeed, the process of triggering the germination of dormant bacterial spores is fundamentally driven by vital nutrients, including sugars, nucleosides, and amino acids. Nutrient germinants bind to receptors in the inner-membrane of the spore to initiate germination. Additionally, sugars have been shown to increase the binding affinity of L-amino acids for their cognate receptors.

The germinant signal then initiates a cascade that causes the release of Dipicolinic Acid (DPA), which is stored in a 1:1 ratio with Ca²⁺ (CaDPA) in the core of the spore. The release of CaDPA is a fast process and is typically >90% complete in 2 min. CaDPA release represents a point of no return for spores in which they are committed to the germination process. Those knowledgeable in the art refer to this step as the “commitment” step.

After CaDPA release, the spore is partially hydrated and the core pH rises to approx. 8.0. The core of the spore then expands and the cortex (composed mostly of peptidoglycan) is degraded by core lytic enzymes. The spore absorbs water and consequently loses its refractivity. This loss of refractivity towards the end of the germination process allows spore germination to be monitored via phase-contrast microscopy.

The second phase of germination is an outgrowth step in which the spore's metabolic, biosynthetic, and DNA replication/repair pathways initiate. The outgrowth period has several phases. The first is known as a ripening period in which no morphological changes (such as cell growth) occur, but the spore's molecular machinery (e.g. transcription factors, translation machinery, biosynthesis machinery, etc.) is activated. This period can vary in length based on the initial resources that are packaged with the spore during the process of sporulation. For instance, the preferred carbon source of several Bacillus species (including subtilis) is malate and Bacillus spores typically contain a large pool of malate that is used during the revival process. Interestingly, deletion mutants that cannot utilize the malate pool display an extended ripening period compared to wild-type spores indicating that the spore malate pool is sufficient to energize the initial outgrowth process. Additionally, spores store small, acid-soluble proteins that are degraded within the first several minutes of revival that serve as an immediate source of amino acids for protein synthesis. After the outgrowth step, spore revival is complete and cells are considered to be vegetatively growing.

It is known that spores can be induced to germinate via heat-activation. Spores of various Bacillus species have been heat-activated at strain-specific temperatures. For example, B. subtilis spores have been heat-activated at 75° C. for 30 minutes while B. licheniformis spores have been heat-activated at 65° C. for 20 minutes. The heat-activation has been shown to cause a transient, reversible unfolding of spore coat proteins. Heat-activated spores can then be germinated for additional time in germination buffers containing nutrient germinants, such as L-alanine. If no nutrient germinant is present, however, spores will return to their pre-heated, non-germinated state.

It is also known that germination can occur at ambient temperatures (near typical room temperature) without heat-activation and with a germination buffer containing nutrients, but the process usually takes longer than with heat-activation. For example, B. licheniformis and B. subtilis spores will germinate at 35° C. or 37° C., respectively, but it takes a longer period of time (e.g., 2 hours) in a germination buffer containing nutrient germinants. Additionally, non-heat-activated spores of B. subtilis have been known to have been germinated in non-nutrient germinant conditions (e.g., CaCl₂)+Na₂DPA) for an extended period of time.

It is also known to combine the use of heat activation and a nutrient germinant to germinate spores in a two-step process in laboratory settings. The spores are first heat activated by incubating for a period of time (e.g., 30 minutes) at a temperature in the range of 65-75° C. (this specific temperature is species dependent). Then, the spores are transferred into a buffer solution that contains a nutrient germinant, such as L-alanine. It is also known to grow bacteria in a growth chamber located near a use site by feeding pelletized nutrient material (containing sugar, yeast extract, and other nutrients that are not direct spore germinants), bacteria starter, and water into a growth chamber at a controlled temperature range of 16-40° C., and more preferably between 29-32° C., for a growth period of around 24 hours as disclosed in U.S. Pat. No. 7,081,361.

It is also known to germinate spores in a single-step process using specific nutrient germinants and heating at a point of use, without requiring heat activation, as described in U.S. Pat. No. 10,610,552. The nutrient germinant composition disclosed in the '552 patent includes L-amino acids (preferably L-alanine, L-asparagine, L-valine, and/or L-cysteine), buffers (preferably a phosphate buffer, HEPES, and/or Tris base), and an industrial preservative. The nutrient-germinant composition is mixed with spores of Bacillus species and heated to a temperature of 35 to 60 C for an incubation period of 2 to 60 minutes at or near a point of use.

The process and specific nutrient germinants disclosed in the '552 patent work well for most Bacillus spores. However, there exists a subset of Bacillus spores that are wild-type and/or known as “super-dormant spores” that exhibit exceptionally high resistance to germination. Applicant has unexpectedly found that all the Bacillus strains in its repository are wild-type and have shown unresponsiveness to a germination medium containing L-alanine and L-asparagine and using other ingredients and method steps disclosed in the '552 patent. This is unexpected because L-alanine is a significant promoter of Bacillus species germination, with previous research having established that germination is primarily triggered by L-alanine, although further investigations have revealed its dependence on the GerA receptor for efficient germination. This is also unexpected because the compositions and methods of the '552 patent work well for other Bacillus species that Applicant has tested.

While L-alanine is generally considered to be a good germinant, other amino acids have been found to result in poor germination percentages. For example, some studies suggest that branched-chain amino acids like isoleucine, leucine, and valine exhibit poor co-germinant properties, even when incubated at 37° C. Still other studies have found specific combinations of amino acids can have a synergistic effect on germination. For example, it has been shown that, when combined with taurocholate certain amino acids, particularly L-alanine, can synergize to induce spore germination of Clostridium perfringens type A and G strains by approximately 50%.

The inactivity observed in Applicant's super-dormant wild-type spores poses a significant challenge. These strains, which have undergone thorough screening, remain unusable due to their inability to germinate using known nutrient germinant compositions and methods. Thus, there is a need for a spore activation composition and method that will allow generation of germinated or activated Bacillus species, particularly super-dormant and wild-type species, and can preferably result in at least 50% germination within 1 hour.

### SUMMARY OF THE DISCLOSURE

A nutrient-germinant solution according to one preferred example of the disclosure useful for activating or germinating wild-type and/or super-dormant bacteria spores, particularly Bacillus spores, comprises the amino-acids L-isoleucine or L-Valine or both. A nutrient-germinant solution in some preferred examples comprises 15 to 40 g/L L-isoleucine. A nutrient-germinant solution according to some preferred examples comprises 30 to 85 g/L L-valine. When both amino acids are used in some preferred examples, a nutrient-germinant solution may comprise 70 to 125 g/L total of the L-isoleucine and L-valine. In such examples, the L-isoleucine and L-valine may be in a ratio of around 1.5 to 4:3 to 8.5, more preferably around 2.5 to 3.5:4 to 7, and most preferably around 3.5 to 4:7 to 8.5.

In other preferred examples, a nutrient-germinant solution further comprises one or more other L-amino acids or similar compounds, such as aspartic acid or inosine. When other L-amino acids are used, a nutrient-germinant solution may comprise around 62 to 159 g/L total of the other L-amino acids, in addition to the amounts of L-isoleucine and/or L-valine. In some preferred examples, one or more other L-amino acids used with L-isoleucine or L-valine in a nutrient-germinant solution may comprise one or more of L-arginine, L-tryptophan, L-tyrosine, L-phenylalanine, L-cysteine, L-cystine, L-glutamine, L-proline, and L-lysine. In some examples, aspartic acid and/or inosine may also be combined with these one or more L-amino acids in a nutrient-germinant composition.

In still other preferred examples, a nutrient-germinant solution does not comprise any L-amino acids other than L-isoleucine and/or L-Valine. In still other preferred examples, a nutrient-germinant solution does not comprise any aspartic acid or inosine.

According to some preferred examples, a nutrient-germinant solution further comprises one or more other ingredients in addition to L-amino acids. These other ingredients may comprise: (1) 23 to 45 g/L of a source of potassium, most preferably potassium chloride; (2) 4 to 20 g/L total of one or more buffers; and (3) 405 to 809 g/L water. According to one preferred example, the one or more buffers comprise one or more phosphate buffers, and most preferably disodium phosphate and/or monosodium phosphate.

According to another preferred examples, a liquid nutrient-germinant composition or a liquid nutrient-spore composition further comprises 0.5-3.3 g/L of an industrial preservative to prevent growth of background contaminants during shipping and storage. Industrial preservatives may comprise methyl chloro isothiazolinone and/or methyl isothiazolinone (such as in the commercially available Kaython™ CG/ICP or Kathon™ CG/ICP II or Linguard ICP™), propylparaben, and/or methylparaben. A germination inhibitor, such as salt (NaCL) and/or D-alanine, may also be used as an alternative to, or in addition to, an industrial preservative. For a liquid nutrient-spore composition, it is preferred to include both an industrial preservative and a germination inhibitor.

In still other preferred examples, a nutrient-germinant solution comprises (1) water, (2) around 1 ml/L Kathon (an industrial preservative), (3) around 44.7 g/L potassium chloride, (4) around 5 g/L monosodium phosphate, (5) around 15 g/L disodium phosphate, and (6) one or more L-amino acids, wherein the one or more L-amino acids comprise: (a) 30 g/L L-isoleucine, (b) 40 g/L L-isoleucine, (c) 60 g/L L-valine, (d) 85 g/L L-valine, (e) 40 g/L L-isoleucine and 85 g/L L-valine, or (f) any one of the combinations of L-amino acids listed in Table 2 herein.

In still other preferred examples, a nutrient-germinant solution comprises (1) water, (2) 1 ml/L Kathon (an industrial preservative), (3) 45 g/L potassium chloride, (4) 10 g/L monosodium phosphate, (5) 15 g/L disodium phosphate, and (6) one or more L-amino acids, wherein the one or more L-amino acids comprise: (a) 30 g/L L-isoleucine, (b) 40 g/L L-isoleucine, (c) 60 g/L L-valine, (d) 85 g/L L-valine, (e) 40 g/L L-isoleucine and 85 g/L L-valine, or (f) any one of the combinations of L-amino acids listed in Table 2 herein.

Preferred examples of germinant compositions herein include nutrient-germinant compositions in liquid (concentrated or diluted) or dry forms that may be mixed with spores or a spore composition comprising wild-type and/or super-dormant spores, preferably of Bacillus, at a point-of-incubation and at or near a point-of-use. Preferred examples of germinant compositions herein also include nutrient-germinant compositions that are pre-combined or pre-mixed with spores or a spore composition comprising wild-type and/or super-dormant spores, preferably of Bacillus, in liquid (concentrated or diluted) or dry forms. Dry forms of nutrient-germinant compositions and/or nutrient-spore compositions may be mixed with water at a point-of-incubation and at or near a point-of-use to form a nutrient-spore solution for incubation.

According to another preferred example, wild-type and/or super-dormant spores are pre-activated prior to incubation, and most preferably at the manufacturer prior to packing for shipment or storage. In one preferred example, a pre-activation method comprises (1) combining a liquid nutrient-germinant composition according to a preferred example herein with spores near an end of a fermentation process for the spores to form a mixed liquid solution, (2) bioprocessing the mixed liquid solution for a bioprocessing period of time of 10-30 minutes, and then (3) drying the mixed liquid solution to form a dry nutrient-spore composition comprising pre-activated spores. In another preferred example, a pre-activation method comprises (1) mixing a buffer solution (preferably a phosphate buffer) with the spores (after completion of a fermentation process for the spores) in a container, (2) adding a surface area enhancement media to the container and shaking or stirring, (3) adding a surfactant to the container, (4) adding a nutrient-germinant composition according to a preferred example herein (preferably in liquid form) to the container to form a mixed liquid solution, (5) heating the mixed liquid solution to a temperature in a range of 35 to 45° C. for a heat treatment period of time of 180 seconds to 10 minutes, (6) placing the heated mixed liquid solution in an ice bath for at least 180 seconds, and (7) drying the cooled mixed liquid solution to form a dry nutrient-spore composition comprising pre-activated spores.

According to one preferred example, a method of drying either nutrient-germinant composition, a nutrient-spore composition, or a mixed liquid solution comprises spray drying at a temperature in a range of around 117 to 130° C. to form a dry nutrient-germinant composition, a dry nutrient-spore composition, a dry nutrient-spore composition with pre-activated spores. Other forms of drying, such as freeze-drying, air drying, or drum drying may also be used. Dry compositions herein preferably comprise less than 4%, more preferably less than 2%, and most preferably less than 0.1% water.

Preferred methods of incubation, or activation or germination, herein comprise heating a nutrient-spore solution according to an example herein to a temperature in a range of 26 to 60° C. for an incubation period. A nutrient-spore solution used in incubation methods according to preferred examples may comprise spores that have been pre-activated using a pre-activation method according to a preferred example herein or any other heat shock or pre-activation method. A nutrient-spore solution used in incubation methods according to preferred examples may comprise spores that have not been pre-activated using a pre-activation method herein or any other heat shock or pre-activation method. In some preferred examples, an incubation period may be 30 mins to 36 hours, more preferably 45 mins to 24 hours, and most preferably 1 hour to 6 hours. Incubation methods herein are capable of producing an incubated bacteria solution comprising activated or fully germinated bacteria, depending on the species and the incubation period.

Germinant compositions and incubation methods according to preferred examples herein are useful in activating or germinating super-dormant and wild-type species bacteria species, particularly Bacillus species, that are not capable of activation or have lower activation rates when using prior art germinants and incubation methods. Incubation methods using germinant composition according to preferred examples are capable of achieving spore activation rates of at least 50%, more preferably at least 70%, and most preferably at least 90% of these wild-type and/or super-dormant bacteria spores after 24 hours. When an incubation temperature is around 40 to 42° C. (or higher), an activation rate of at least 50% may be achievable for most wild-type and/or super-dormant Bacillus spore strains in incubation periods as short as 1 hour. These activation rates may be achieved with a single step incubation process, where the spores are not pre-activated or heat shocked. Even higher rates of activation can be achieved with a two step process, where pre-activated spores are combined with an incubation method according to a preferred example.

### BRIEF DESCRIPTION OF THE DRAWINGS

The disclosure is further described and explained in relation to the following drawings wherein:

FIGS. 1A-1B are photographs of bacteria slides of STRAIN-1 using certain nutrient-germinant compositions in Example 1 after incubating at 42° C. for 1 hour and for 24 hours;

FIGS. 2A-2B are photographs of bacteria slides of STRAIN-1 using certain nutrient-germinant compositions in Example 2 after incubating at 42° C. for 1 hour and for 24 hours;

FIG. 3 are photographs of bacteria slides of STRAIN-1 comparing a nutrient-germinant composition according to a preferred example with two control compositions in Example 3 after incubating at 42° C. for 1 hour and for 24 hours;

FIG. 4A-4B are photographs of bacteria slides of various strains of wild-type super-dormant Bacillus using a nutrient-germinant composition according to one preferred example in Example 4 after incubating at 42° C. for 1 hour and for 24 hours;

FIG. 4C-4D are photographs of bacteria slides of various strains of wild-type super-dormant Bacillus using a nutrient-germinant composition according to one preferred example in Example 4 after incubating at 35° C. for 1 hour and for 24 hours;

FIG. 4E-4F are photographs of bacteria slides of various strains of wild-type super-dormant Bacillus using a nutrient-germinant composition according to one preferred example in Example 4 after incubating at 25° C. for 1 hour and for 24 hours;

FIG. 5 are photographs of bacteria slides of STRAIN-1 comparing nutrient-germinant compositions according to a preferred examples in Example 5 after incubating at 42° C. for 1 hour and for 24 hours;

FIG. 6 are photographs of bacteria slides of STRAIN-1 pre-activated according to a preferred example and mixed with a nutrient-germinant composition according to a preferred example as compared to STRAIN-1 that has not been pre-activated, after mixing each with water and prior to any incubation in Example 6; and

FIGS. 7A-7C are tables of various combinations of L-amino acids that were tested as described in Example 2.

### DESCRIPTION OF THE PREFERRED EXAMPLES

### Nutrient-Germinant Compositions

A nutrient-germinant composition according to one preferred example of the disclosure comprises the amino acids L-isoleucine and/or L-Valine. A nutrient-germinant composition according to preferred examples of the disclosure may be in a liquid form or may be in a solid or dry form. As used herein, references to a liquid nutrient-germinant composition (or similar wording, such as a nutrient-germinant composition in liquid form) refer to a pre-mixed liquid nutrient-germinant composition that is shipped from a manufacturer as a liquid, solution, or slurry (concentrated or diluted as a ready-to-use working solution). As used herein, references to a dry nutrient-germinant composition (or similar wording, such as a nutrient-germinant composition in dried or powdered form) refer to a pre-mixed dry nutrient-germinant composition that is shipped from a manufacturer in solid (such as pellets or brick) or powdered forms that are substantially free of water, comprising less than 2% water. As used herein, references to a nutrient-germinant solution refer to (1) a liquid nutrient-germinant composition, preferably having ingredients within concentration ranges indicated herein or (2) a dry nutrient-germinant composition that has been mixed with water, preferably in amounts to provide the concentration ranges indicated herein and preferably mixed at a point-of-incubation and near a point-of-use.

A nutrient-germinant composition according to one preferred example preferably comprises 15 to 40 g/L, more preferably 25 to 35 g/L, and most preferably 35 to 40 g/L L-isoleucine. A nutrient-germinant solution according to another preferred example comprises 30 to 85 g/L, more preferably 40 to 70 g/L, and most preferably 70 to 85 g/L L-valine.

A nutrient-germinant composition according to another preferred example comprises both L-isoleucine and L-valine. When both amino acids are used, a nutrient-germinant solution preferably comprises 70 to 125 g/L, more preferably 105 to 125 g/L total of the L-isoleucine and L-valine. Preferably, the L-isoleucine and L-valine are in a ratio of around 1.5 to 4:3 to 8.5, more preferably around 2.5 to 3.5:4 to 7, and most preferably around 3.5 to 4:7 to 8.5.

In other preferred examples, a nutrient-germinant composition further comprises one or more other L-amino acids and/or Inosine, in addition to L-isoleucine and/or L-valine. Other L-amino acids that may be included in some examples comprise L-alanine, L-asparagine, and L-cysteine. In a further example, L-amino acids can be provided as a hydrolysate of soy protein. When other L-amino acids are used, a nutrient-germinant solution preferably comprises around 62 to 159 g/L, more preferably around 87 to 133 g/L, and most preferably around 133 to 159 g/L total of the other L-amino acids, in addition to the amounts of L-isoleucine and/or L-valine.

In still other preferred examples, a nutrient-germinant composition does not comprise any L-amino acids other than L-isoleucine or L-Valine. In still other preferred examples, a nutrient-germinant composition does not include any L-Alanine, L-asparagine, L-glutamic acid, L-histidine, and/or Inosine.

Nutrient-germinant compositions according to some preferred examples are in a liquid form. Nutrient-germinant composition according to some preferred examples may comprise one or more other ingredients in addition to L-amino acids. These other ingredients may comprise: (1) 23 to 45 g/L, more preferably 28 to 45 g/L, and most preferably 39 to 45 g/L of a source of potassium, most preferably potassium chloride; (2) 4 to 20 g/L, more preferably 12 to 17 g/L, and most preferably 13 to 15 g/L total of one or more buffers, preferably one or more phosphate buffers; and (3) 405 to 809 g/L, more preferably 406 to 809 g/L, and most preferably 708 to 809 g/L water. According to another preferred example, a nutrient-germinant composition may comprise a source of phosphates, particularly if the one or more buffers are not phosphate buffers.

In some preferred examples, a nutrient-germinant composition comprises at least two phosphate buffers, most preferably disodium phosphate and monosodium phosphate. Amounts of disodium phosphate used are preferably 7.5 to 15 g/L, more preferably 9 to 15 g/L, and most preferably 13 to 15 g/L. Amounts of monosodium phosphate used are preferably 2.5 to 5 g/L, more preferably 3 to 5 g/L, and most preferably 4 to 5 g/L.

A liquid nutrient-germinant composition preferably further comprises an industrial preservative. The inclusion of a general, industrial preservative in the composition aids in long-term storage and/or germination inhibition, which is particularly useful when the composition is in a preferred concentrated liquid form. An industrial preservative may aid in preventing growth of background contamination that may be present in a nutrient-germinant composition during shipment and storage prior to use at a point-of-use. The end use location may include agricultural settings where air conditioned storage is not available. Without the preservative, any background bacterial or fungal contamination contained in a nutrient-germinant composition may germinate and begin vegetative growth during storage conditions, and particularly under the higher heat ambient conditions encountered in agricultural settings that can easily reach over 37° C. This would render the nutrient-germinant composition unusable for activating or germinating the desired Bacillus species as the nutrient ingredients would be exhausted. Salmonella, Pseudomonas, and Streptomyces are examples of bacteria that are known to utilize one or more L-amino acids, such as alanine and arginine, as carbon sources for growth. The preservative prevents such bacteria from utilizing the L-amino acids in the nutrient-germinant composition as a source of carbon to prevent contamination growth, allowing a nutrient-germinant composition according to some preferred examples to be stored at a point of use without negatively impacting its usefulness.

A liquid nutrient-germinant composition preferably comprises around 0.5-3.3 g/L, more preferably 1.2-2.7 g/L, most preferably 1.6-2.2 g/L total of one or more industrial preservatives. Preferred industrial preservatives comprise methyl chloro isothiazolinone and/or methyl isothiazolinone, such as in the commercially available Kaython™ CG/ICP (comprising 1.0 to less than 2.5% of a combination of 5-chloro-2-methyl-4-isothiazolin-3-one and 2-methyl-2H-isothiazol-3-one) or Kathon™ CG/ICP II (comprising more than 1.1 to 1.25% 5-chloro-2-methyl-4-isothiazolin-3-one; more than 0.3 to 0.45% 2-methyl-4-isothiazolin-3-one; less than 0.6% magnesium chloride; more than 1.4% to 2.7% magnesium dinitrate; more than 0.15% to 0.17% copper (II) nitrate; and more than 95.5 to 96.2% water) or Linguard ICP™ (which has up to 1.5% methychloroisothiazolinone and methylisothiazolinone). When Kathon™ CG/ICP or Kathon™ CG/ICP II is used, an amount according to one preferred example is 0.5 to 1 g/L, more preferably 0.63 to 0.88 g/L, and most preferably 0.88 to 1 g/L for Kathon. Other preservatives that may be used according to other preferred examples comprise propylparaben or methylparaben. A germination inhibitor, such as salt (NaCL) and/or D-alanine, may also be used as an alternative to, or in addition to, an industrial preservative, in some preferred examples of nutrient-germinant compositions. Most preferably, any preservative or germination inhibitor is GRAS (Generally Regarded As Safe under U.S. federal standards). A liquid nutrient-germinant composition preferably comprises around 29-117 g/L, more preferably 43-88 g/L, most preferably 52-71 g/L of NaCL and/or around 8-116 g/L, more preferably 26-89 g/L, most preferably 40-50 g/L of D-alanine, if a germination inhibitor is included.

In some preferred examples, a dry nutrient-germinant composition may be in a solid form such as pellets or bricks or powder. A dry nutrient-germinant composition may be mixed with water, preferably DI water, to form a nutrient-germinant solution, which is mixed with spores for activation according to a preferred method of incubating spores herein. A dry nutrient-germinant composition preferably comprises less than 4%, more preferably less than 2%, and most preferably less than 0.1% water. According to one preferred example, dry nutrient-germinant compositions comprise the same ingredients in proportionate amounts as in preferred examples of a liquid nutrient-germinant composition described herein, except that it may not be necessary to use a preservative or germination inhibitor with a dry nutrient-germinant compositions formulations.

Most preferably, nutrient-germinant compositions are in a liquid form, and particularly in a concentrated liquid form, which is easier and faster to mix with diluent at the point-of-use than a dry nutrient-germinant composition. When in a concentrated form, a liquid nutrient-germinant composition is diluted with water or any other appropriate diluent, preferably at the point-of-use, to form a nutrient-germinant solution. Concentration amounts indicated herein are for a nutrient-germinant solution. The diluted concentrations in a nutrient-germinant solution are preferably in a range from 0.1-10.0% of the concentrated concentrations, but other amounts may also be used. The use of a concentrated liquid nutrient-germinant composition reduces shipping, storage, and packaging costs and makes dosing of a liquid nutrient-germinant composition at the point-of-use easier.

The amounts of these ingredients are important aspects of the disclosure because higher concentrations would render some ingredients insoluble and lower concentrations would be ineffective at activating or germinating spores.

### Nutrient-Spore Compositions

According to another preferred example, a nutrient-germinant composition may further comprise spores. A nutrient-germinant composition comprising spores is referred to herein as a nutrient-spore composition. A nutrient-spore composition may be shipped from a manufacturer as a pre-mixed combination of a nutrient-germinant composition and spores or may be formed at a point-of-incubation and near a point-of-use by mixing a nutrient-germinant composition and spores. A nutrient-spore composition may be in a liquid form or may be in a solid or dry form (such as a powder, as pre-mixed and shipped from a manufacturer). As used herein, references to a liquid nutrient-spore composition (or similar wording, such as a nutrient-spore composition in liquid form) refer to a pre-mixed liquid nutrient-spore composition that is shipped from a manufacturer as a liquid, solution, or slurry (concentrated or diluted as a ready-to-use working solution). A liquid nutrient-spore composition preferably comprises (1) around 55 to 95% w/v, more preferably around 70 to 85%, and most preferably around 74 to 83% of a nutrient-germinant composition and (2) around 5 to 13% w/v, more preferably around 9 to 11%, and most preferably around 9.5 to 10.5% of a spore composition (preferably comprising 40-60% spores by weight, but may comprise more or less spores). Ingredients in a nutrient-germinant composition used in a liquid nutrient-spore composition are preferably in the same amounts as a nutrient-germinant solution according to preferred examples herein.

As used herein, references to a dry nutrient-spore composition (or similar wording, such as a nutrient-spore composition in dried or powdered form) refer to a pre-mixed dry nutrient-spore composition that is shipped from a manufacturer in solid (such as pellets or brick) or powdered forms that are substantially free of water, comprising less than 4% water. A dry nutrient-spore composition preferably comprises (1) around 10 to 90% and more preferably around 40 to 60%, and most preferably around 40 to 50% by weight of a nutrient-germinant composition and (2) around 10 to 90%, more preferably around 30 to 60%, and most preferably around 40 to 50% by weight of a spore composition (preferably comprising 40-60% spores by weight, but may comprise more or less spores). Ingredients in a nutrient-germinant composition used in a dry nutrient-spore composition are preferably in the same amounts or comparable ratios when dried as a liquid nutrient-germinant solution according to preferred examples herein.

As used herein, references to a nutrient-spore solution refer to (1) a liquid nutrient-spore composition, or (2) a dry nutrient-spore composition that has been mixed with water, preferably at a point-of-incubation and near a point-of-use, or (3) a nutrient-germinant solution that has been mixed with spores, preferably at a point-of-incubation and near a point-of-use, to form a nutrient-spore solution. Most preferably, a nutrient-spore solution comprises concentrations of a nutrient-germinant composition and spores as indicated above for a liquid nutrient-spore composition and the individual ingredients in the nutrient-germinant composition therein are preferably in the same concentrations as indicated above for a nutrient-germinant solution.

Spores included in a nutrient-spore composition may be part of a separate spore composition that is added and mixed with a nutrient-germinant composition to form a nutrient-spore composition. A spore composition preferably comprises spores and salt (table salt), most preferably around 60-40% bacteria spores and around 40-60% salt (table salt). Other amounts of spores may also be used. Most preferably, a spore composition is used to make a nutrient-spore composition in amounts needed to provide a concentration or weight percentage of spores as indicated above.

In other preferred examples, a liquid nutrient-spore composition further comprises one or more germination inhibitors (in addition to an industrial preservative or as an alternative to an industrial preservative. Preferred germination inhibitors include NaCl and/or D-alanine. A liquid nutrient-spore composition preferably comprises around 29-117 g/L, more preferably 43-88 g/L, most preferably 52-71 g/L of NaCL and/or around 8-116 g/L, more preferably 26-89 g/L, most preferably 40-50 g/L of D-alanine. These germination inhibitors aid in maintaining the spores in an inactive state and prevent premature germination of spores prior incubation according to methods herein at a point-of-use. It is not necessary to include a germination inhibitor in a dry nutrient-spore composition.

According to another preferred example, a dry nutrient-spore composition is formed by mixing a nutrient-germinant composition (preferably one according to an example herein and preferably a nutrient-germinant solution) with wild-type and/or super-dormant spores to form a mixed liquid solution and then drying the mixed liquid solution. Most preferably, a mixed liquid solution is the same as or comprises a liquid nutrient-spore composition. Other ingredients or components may also be included in a mixed liquid solution, such as ingredients used in a fermentation process for preparing the spores, buffers, and/or surfactants.

A mixed liquid solution according to preferred examples herein is dried, preferably by spray drying, at a temperature in a range of around 117 to 130° C., more preferably around 119 to 128° C., and most preferably around 117 to 122° C. to form a dry nutrient-spore composition. Although spray-drying is preferred, freeze-drying, air drying or drum drying may also be used. A dry nutrient-spore composition preferably comprises less than 4%, more preferably less than 2%, and most preferably less than 0.1% water.

According to one preferred example, a nutrient-germinant composition may be adsorbed to or absorbed by the spores in the mixed liquid solution or during the drying to form a dry nutrient-spore composition. According to another preferred example, ingredients in a nutrient-germinant composition and the spores may remain separate and dispersed throughout the dry nutrient-spore composition, preferably microscopically dispersed so that individual particles consisting essentially of bacteria spores and individual particles consisting essentially of ingredients from a nutrient-germinant composition are not visible to the naked eye. In still other preferred examples, a combination of adsorption, absorption, and/or dispersion of separate, individual particles may be included in a dry nutrient-spore composition.

When forming a mixed liquid solution, a nutrient-germinant composition and spores are preferably not mixed together under conditions which would allow the spores to germinate, as this could cause premature germination with an adverse effect upon the storage life and usability of the dry nutrient-spore composition. For example, according to an alternate example, a mixed liquid solution is not formed, rather a nutrient-germinant composition and spores are kept separate and only combined at the time of or immediately prior to drying such as by using separate streams in a spray-dryer, either by using two nozzles or a single nozzle which permits the simultaneous spraying of two separate streams; or by freeze-drying under conditions (for example temperatures) which are not conducive to germination. Premature germination may also be avoided by introducing spores to a nutrient-germinant composition immediately prior to drying.

Compositions with Pre-Activated Spores

According to another preferred example, a dry nutrient-spore composition comprising pre-activated spores is further formed by bioprocessing a mixed liquid solution prior to drying. As used herein, “pre-activated” means the spores have completed the “commitment” step toward germination. In one preferred example comprising bioprocessing, a nutrient-germinant composition (preferably one according to a preferred example herein) is added to a fermented broth comprising spores towards the end of a fermentation process. A fermentation process is preferably one as described below with respect to the examples herein, but other known fermentation processes may also be used. A liquid nutrient-germinant composition according to a preferred example is preferably added at a final mixing step of the fermentation to form a mixed liquid solution, preferably having amounts and/or ratios of nutrient-germinant composition ingredients and spores as previously indicated for a liquid nutrient-spore composition. The mixed liquid solution is then bioprocessed by mixing the entire solution according to protocol in a closed Bio-flow chamber for a bioprocessing period of time, preferably of at least 10 minutes, more preferably at least 15 minutes, and most preferably for at least 25 minutes. This final step preferably does not continue longer than 30 minutes. After bioprocessing is completed, a mixed liquid solution comprises pre-activated spores with light blue appearance compared to inactivated spores that appear bright under the phase contrast microscope. The mixed liquid solution is then dried as previously described to form a dry nutrient-spore composition comprising pre-activated spores.

In another preferred example, a dry nutrient-spore composition comprising pre-activated spores is further formed by heat treating a mixed liquid solution prior to drying. In one preferred example comprising heat treating, a nutrient-germinant composition (preferably one according to a preferred example herein) is mixed with spores after a fermentation process is complete. Again, a fermentation process is preferably one as described below with respect to the examples herein, but other known fermentation processes may also be used. In this preferred example, spores are added to a phosphate buffer solution in a container and mixed thoroughly. A phosphate buffer solution preferably comprises sodium phosphate dibasic heptahydrate and sodium phosphate monobasic monohydrate (preferably 2.02% w/v and 0.34% w/v respectively). Preferably, around 50 to 250 mL, more preferably around 80 to 200 mL, and most preferably around 200 to 250 mL of a phosphate buffer solution is used per 1 to 5 g of spores.

Next, a surface area enhancement media, such as glass beads, along with a surfactant are added to the container and shaken or stirred until well mixed. Most preferably, the surfactant is a non-ionic surfactant, such as Tween. Preferably, around 1 to 5 mL, more preferably around 2 to 4 mL, and most preferably around 4 to 5 mL of a surfactant is used per 1 to 5 g of spores. The surfactant composition is preferably around 0.25 to 1.25% by weight of the final mixture that is dried.

Then, a nutrient-germinant composition is added to the container to form the mixed liquid solution, preferably having amounts and/or ratios of nutrient-germinant composition ingredients and spores as previously indicated for a liquid nutrient-spore composition. The mixed liquid solution is then heated, preferably in a water bath, to a temperature in a range of 35 to 45° C., more preferably 37 to 40° C., and most preferably 40 to 42° C. for a heat-treatment period of time, preferably of at least 180 seconds, more preferably at least 300 seconds, but most preferably for no more than 10 minutes. The mixed liquid solution was then transferred into a beaker and immediately cooled in an ice bucket for preferably of at least 180 seconds, more preferably at least 300 seconds. The cooled mixed liquid solution is then dried as previously described to form a dry nutrient-spore composition comprising pre-activated spores.

### Methods of Activating or Germinating Spores

Nutrient-germinant compositions or nutrient-spore compositions according to preferred examples are useful in activating or germinating wild-type and/or super-dormant bacteria spores, particularly Bacillus spores, but it can also be used with other genera of bacteria.

According to one preferred example, a method of incubating or activating or germinating wild-type and/or super-dormant bacteria spores comprises: incubating a nutrient-spore solution, preferably according to preferred examples herein, by heating to a temperature in a range of 26 to 80° C., more preferably 35 to 70° C., and most preferably 40 to 50° C. for an incubation period to form an incubated bacteria solution. According to another preferred example, incubation make take place at room temperature of around 23 to 25° C., but incubation is preferably by heating to above 25° C. An incubation period is preferably 30 mins to 36 hours, more preferably 45 mins to 24 hours, and most preferably 1 hour to 6 hours. Heating of a nutrient-spore solution during the incubation period takes place with the spores and nutrient-germinant composition ingredients together, preferably in a single step. Depending on the end-use application, an incubation period may be shorter (to produce activated, but not fully germinated bacteria that stand a better chance of surviving through to an ingesting animal's intestinal tract where they are most beneficial) or longer (to produce fully vegetative bacteria). The incubation step may be in an air incubator, a water incubator, or any other chamber that provides even, constant heat at the given temperature range.

According to one preferred example, a nutrient-spore solution used in methods herein comprises spores that have not been heat shocked or pre-activated, but are heated for the first time during the incubation step, in a single step process. According to another preferred example, a nutrient-spore solution used in methods herein comprises spores that have been heat shocked or pre-activated, such as in a dry nutrient-spore composition with pre-activated spores, prior to the incubation step, in a two-step process (pre-activation combined with incubation).

According to another preferred example, a method further comprises forming a nutrient-spore solution prior to the incubating step by adding a nutrient-germinant solution to wild-type and/or super-dormant bacteria spores and mixing to form the nutrient-spore solution. According to another preferred example, a method further comprises forming a nutrient-spore solution prior to the incubating step by adding a dry nutrient-spore composition and water, preferably DI water, and mixing to form the nutrient-spore solution. According to still another preferred example, the dry nutrient-spore composition is one that comprises pre-activated spores. Most preferably, the nutrient-spore solution formed by these examples comprises a nutrient-spore solution having the concentrations of ingredients according to preferred examples herein.

According to another preferred example, a method further comprises forming a nutrient-germinant solution prior to the forming a nutrient-spore solution step by adding a diluent, preferably comprising water and more preferably comprising DI water, to a concentrated liquid nutrient-germinant composition or to a solid nutrient-germinant composition. Most preferably, the nutrient-germinant solution formed comprises a nutrient-germinant solution having the concentrations of ingredients according to preferred examples herein.

According to preferred methods herein, an incubated bacteria solution preferably comprises at least 50%, more preferably at least 70%, and most preferably at least 90% activated bacteria. When an incubation temperature is around 23 to 25° C., an activation rate of at least 50% may be achievable for most wild-type and/or super-dormant Bacillus spore strains in incubation periods of around 24 hours and for some of those Bacillus spore strains in incubation periods around 1 hour. When an incubation temperature is around 40 to 42° C. (or higher), an activation rate of at least 50% may be achievable for most wild-type and/or super-dormant Bacillus spore strains in incubation periods of 1 hour.

When a dry nutrient-spore composition with pre-activated spores is used in preferred methods herein, an incubated bacteria solution preferably comprises at least 90%, more preferably at least 75%, and most preferably at least 50% activated bacteria with an incubation period of around 1 hour.

Preferred methods of incubating or activating or germinating wild-type and/or super-dormant bacteria spores take place at or near a point-of-use, such as an animal (through feed or water), animal bedding, plants, ponds, humans, wastewater system, or drain. The method may also preferably comprise the step of dispensing the incubated bacteria solution to the point-of use.

### Examples of Preferred Compositions and Methods

Applicant has isolated and characterized various wild Bacillus species and assessed their activation levels in response to various compositions according to preferred examples of the disclosure using preferred incubation methods and compared to control compositions. A germination medium containing L-alanine (“L-alanine Control Composition”) was used as a positive control. The L-alanine Control Composition is within the scope of a nutrient-germinant composition disclosed in the '552 patent, comprising 845.3 ml/L water, 1 ml/L Kathon (industrial preservative), 89 g/L L-alanine, 44.7 g/L potassium chloride, 5 g/L monosodium phosphate, and 15 g/L disodium phosphate. Some of the assessments also used a phosphate buffer solution (the same sodium phosphate dibasic heptahydrate and sodium phosphate monobasic monohydrate composition used in forming a dry nutrient-spore composition comprising pre-activated spores by heat treating as previously described) as a negative control (“Buffer control composition”).

The compositions were tested on wild-type super-dormant Bacillus spores. The spore strains were prepared for testing using a fermentation process in which organic compounds are broken down and converted into another organic compound and ATP. In return, the number of bacteria multiplies. The fermentation process was the same used for all of the test strains of wild-type Bacillus species in the Examples below.

Spores from each example were observed using phase contrast microscopy. Slides were prepared using standard procedures. Spores were viewed on an Olympus BX41 microscope (100× oil emersion objective) and imaged using an Olympus UC30 camera controlled by the cellSens Dimension software package. Images were taken and germinated or activated spores were counted as a percentage of the total spores in the field. A total of 12 representative images were analyzed for each example sample number per spore tested. Germinated or activated spores lose their refractivity due to the influx of water and are phase-dark (dark blue) while non-germinated spores are phase-bright.

The compositions, methods, and results in the Examples are described below. Each Example was replicated three times with activation rates indicated being averaged over the full data from all three data sets.

### Example 1—Assessment of Individual Amino Acids for Activation of STRAIN-1

Table 1 shows the list of the individual amino acids and their respective concentrations used in these tests. Initially, a well-characterized wild strain, STRAIN-1 (which has not yet been identified as being in a particular species and is a naturally occurring Bacillus) was evaluated prior to testing other wild-type Bacillus strains. STRAIN-1 typically exhibits about 20% activation after 1 hour and 95% activation after 24 hours in our current L-alanine Control composition with incubation at 42° C. Briefly, 1 mg of STRAIN-1 spores was suspended in 1 ml of combined amino acids. Negative control experiments utilized the Buffer Control composition, while positive controls used L-alanine Control composition. Spores were exposed to various germination solutions having the individual amino-acids listed in Table 1 for 1 and 24 hours while heating to a temperature of 42° C. The germinant solution for each test included all of the same ingredients and amounts as the L-alanine Control Composition except for the specific amino acid, which was at the concentrations indicated in Table 1. The varying concentrations of L-amino acids were selected based on the solubility of each amino acid, with higher amounts used for less soluble amino acids to try to standardize the tests. Of these germinant solutions, sample numbers 10 (containing L-isoleucine) and 17 (containing L-valine) are nutrient-germinant composition according to preferred examples of the disclosure.

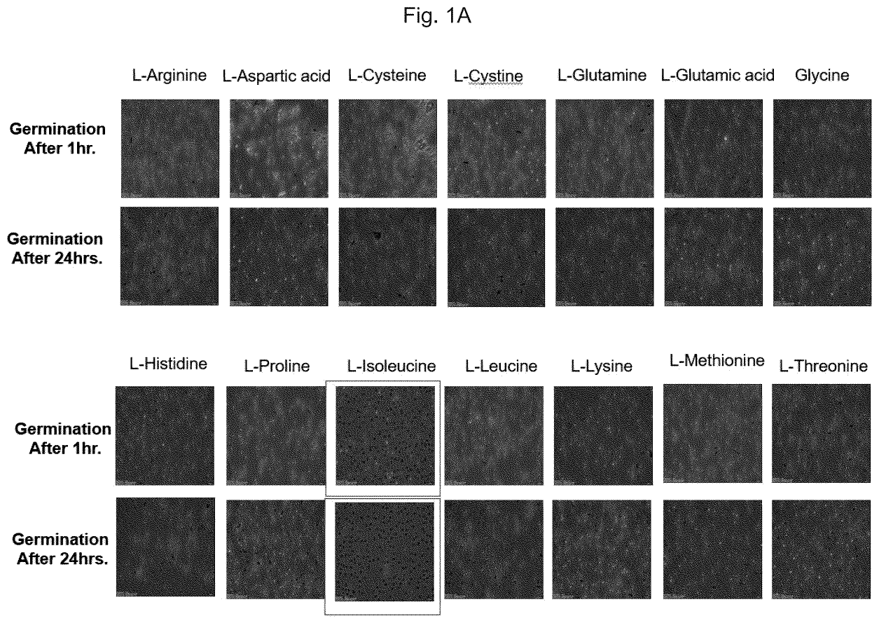

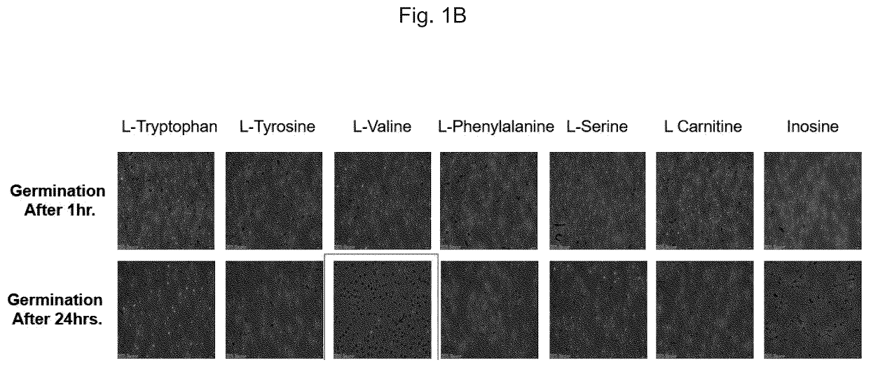

At the end of the 1 and 24 hour periods, 2 μl of the suspended spore was placed on a glass slide, covered with a coverslip, and observed under a phase-contrast microscope. As shown in FIGS. 1A-1B (with images taken after 1 hour of incubation and after 24 hours of incubation) for each germinant solution tested, dormant spores appeared phase-bright, while activated spores appeared phase-dark. The results of these tests are shown below in Table 1.

Although not shown in Table 1, the L-alanine Control Composition and Buffer Control Composition were also tested under the same conditions described above, including heating to 42° C., with the same amount of STRAIN-1 and the same sampling procedure at 1 hour and 24 hours. The L-alanine Control Composition has 89 g/L of L-alanine. The activation rate in the L-alanine Control Composition was 20% after 1 hour and 95% after 24 hours. The activation rate in the Buffer Control Composition was zero after 1 hour and zero after 24 hours.

As can be seen in Table 1, of all the individual amino acids tested with STRAIN-1, only the two that are nutrient-germinant compositions according to preferred examples herein resulted in any activation after 1 hour or after 24 hours. The nutrient-germinant composition with L-isoleucine (sample number 10) resulted in 70+/−0% activation after only 1 hour and 99% activation after 24 hours. These results far exceed the typical results for STRAIN-1 in the L-alanine Control composition. The nutrient-germinant composition with L-valine (sample number 17) did not result in any activation after 1 hour, but 95% activation after 24 hours. At the 24 hour mark, these results were as good as the typical results for STRAIN-1 in the L-alanine Control composition. The results with the nutrient-germinant compositions according to preferred examples were also far better than any of the other germinant solutions tested in Example 1, which resulted in zero percent activation. These results are unexpected given that prior studies have indicated that branched-chain amino acids like isoleucine, leucine, and valine exhibit poor co-germinant properties, even when incubated. The test results in Table 1 for L-leucine are consistent with the prior studies, as L-leucine resulted in zero percent activation even after 24 hours. But the test results were surprisingly good for L-isoleucine and L-valine. L-isoleucine and L-leucine have the same formula, C₆H₁₃NO₂, but they are structurally different, which changes their physiological properties. Isoleucine, contrary to Leucine, is involved with metabolism and energy regulation.

TABLE 1
List of Amino Acids and their concentrations
used for the individual testing.

| | STRAIN-1 | | | |
|---|---|---|---|---|
| | | Amount of | Activation | Activation |
| Sample | Amino | Amino Acid | Rate (%) | Rate (%) |
| No. | Acid Used | Used (g/L) | at 1 hr. | at 24 hrs. |
| 1 | L-Arginine | 15 | 0 | 0 |
| 2 | L-Aspartic acid | 4 | 0 | 0 |
| 3 | L-Cysteine | 10 | 0 | 0 |
| 4 | L-Cystine | 0.08 | 0 | 0 |
| 5 | L-Glutamine | 30 | 0 | 0 |
| 6 | L-Glutamic acid | 8 | 0 | 0 |
| 7 | Glycine | 200 | 0 | 0 |
| 8 | L-Histidine | 40 | 0 | 0 |
| 9 | L-Proline | 160 | 0 | 0 |
| 10 | L-Isoleucine | 40 | 95 +/− 0 | 99 +/− 0 |
| 11 | L-Leucine | 15 | 0 | 0 |
| 12 | L-Lysine | 80 | 0 | 0 |
| 13 | L-Methionine | 50 | 0 | 0 |
| 14 | L-Threonine | 90 | 0 | 0 |
| 15 | L-Tryptophan | 8 | 0 | 0 |
| 16 | L-Tyrosine | 0.4 | 0 | 0 |
| 17 | L-Valine | 85 | 0 | 95 +/− 0 |
| 18 | L-Phenylalanine | 15 | 0 | 0 |
| 19 | L-Serine | 100 | 0 | 0 |
| 20 | L Carnitine | 500 | 0 | 0 |
| 21 | Inosine | 2.68 | 0 | 0 |

### Example 2—Assessment of Combinations of Amino Acids for Activation of STRAIN-1

Based on the results in Example 1, a second set of tests was conducted to determine potential germination-inducing capabilities with combinations of amino acids. Table 2 shows the list of the combinations of amino acids used in these tests. Each combination in Example 2 included L-isoleucine (sample numbers 9 and 10), L-valine (sample numbers 1-6 and 11-16), or both (sample numbers 7, 8, 17, and 18), along with other L-amino acids as listed. The concentrations for each particular L-amino acid in the various combinations in Example 2 were the same as used for that particular L-amino acid in Example 1 (Table 1). Each sample composition was similar to the L-alanine Control Composition except for the L-amino acids and amounts used. Each sample composition also had the had the following ingredients: 1 ml/L Kathon (industrial preservative), 45 g/L Potassium Chloride, 10 g/L Monosodium Phosphate, and 15 g/L Disodium Phosphate. Of these, sample numbers 1 to 18 are each a nutrient-germinant composition according to a preferred example herein.

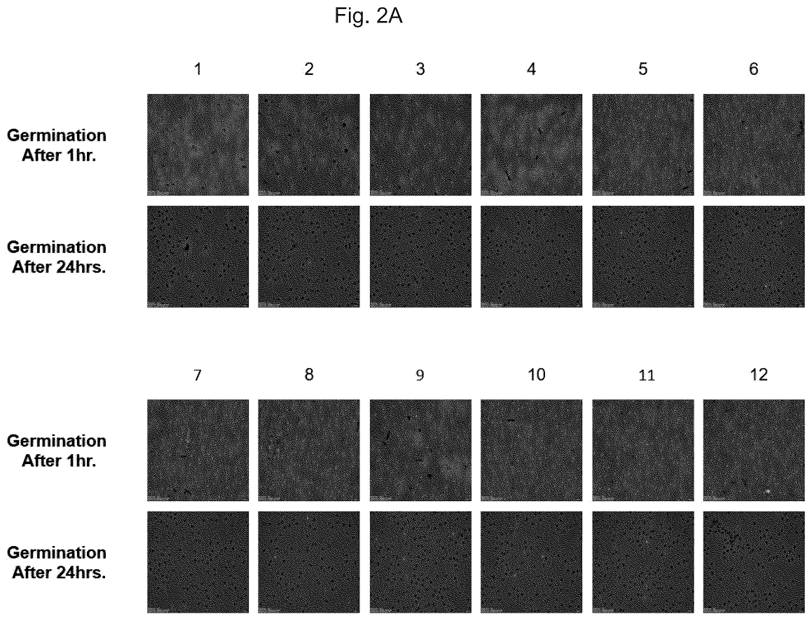

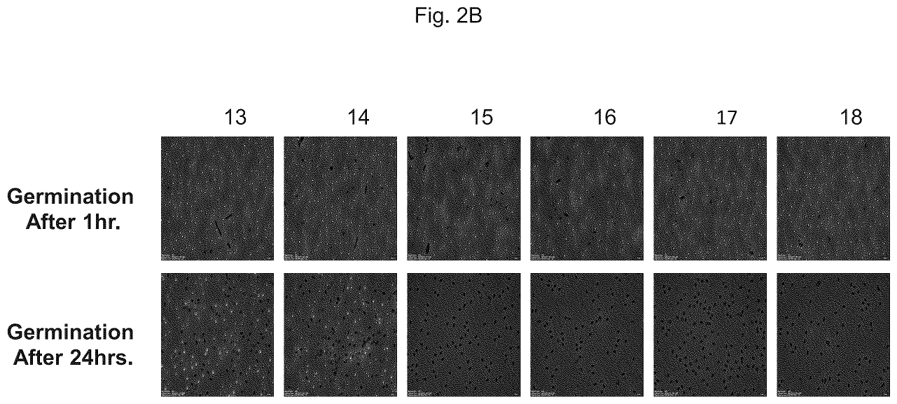

These tests also used STRAIN-1 at the same incubation temperature of 42° C., same amount of STRAIN-1, and the same sampling procedure at 1 hour and 24 hours as used in Example 1. As shown in FIGS. 2A-2B (with images taken after 1 hour of incubation and after 24 hours of incubation) for each germinant solution tested, dormant spores appeared phase-bright, while activated spores appeared phase-dark. The results of these tests are shown below in Table 2.

As can be seen in Table 2, of all the combinations of amino acids tested with STRAIN-1 resulted in activation after 24 hours, but most had no activation after 1 hour. The combinations in sample numbers 5-8, 10-14, and 17-18 all had zero activation after 1 hour. Interestingly, these samples included all of the samples that had both L-isoleucine and L-valine. Without being bound by theory, the poor results in these examples may be due to competition for binding sites. After 24 hours, each of these samples had at least 40% activation, with two of the samples (numbers 17 and 18) that had both L-isoleucine and L-valine being 80% and 90-95%, respectively.

TABLE 2
Amino acid combinations and activation rates.

| | | STRAIN-1 | |
|---|---|---|---|
| | | Activation | Activation |
| Sample | Different Amino | Rate (%) | Rate (%) |
| No. | Acid combinations | at 1 hr. | at 24 hrs. |
| 1 | Arginine, aspartic acid, tryptophan, | 5% | 70% |
| | tyrosine, valine, inosine | | |
| 2 | Arginine, aspartic acid, tryptophan, | 5% | 90 95% |
| | tyrosine, valine | | |
| 3 | Arginine, Tryptophan, tyrosine, | 5% | 90 95% |
| | valine, Phenylalanine, inosine | | |
| 4 | Arginine, Tryptophan, tyrosine, | <5% | 95% |
| | valine, Phenylalanine | | |
| 5 | Cysteine, Cystine, Glutamine, valine, | 0% | 40-60% |
| | Phenylalanine, inosine | | |
| 6 | Cysteine, Cystine, Glutamine, valine, | 0% | 60-80% |
| | Phenylalanine | | |
| 7 | Proline, isoleucine, lysine, valine, | 0% | 40-60% |
| | Phenylalanine, inosine | | |
| 8 | Proline, isoleucine, lysine, valine, | 0% | 40-60% |
| | Phenylalanine | | |
| 9 | Proline, isoleucine, lysine, Tryptophan, | <5% | 40% |
| | tyrosine, inosine | | |
| 10 | Proline, isoleucine, lysine, Tryptophan, | 0% | 40% |
| | tyrosine | | |
| 11 | Tryptophan, tyrosine, valine, inosine | 0% | 80% |
| 12 | Tryptophan, tyrosine, valine | 0% | 90% |
| 13 | Tryptophan, tyrosine, valine, | 0% | 50% |
| | Phenylalanine, Inosine | | |
| 14 | Tryptophan, tyrosine, valine, | 0% | 50-60% |
| | Phenylalanine | | |
| 15 | Arginine, aspartic acid, Tryptophan, | 5-10% | 95-98% |
| | tyrosine, valine, Phenylalanine, inosine | | |
| 16 | Arginine, aspartic acid, Tryptophan, | 5% | 95-98% |
| | tyrosine, valine, Phenylalanine | | |
| 17 | Cysteine, Cystine, Glutamine, Proline, | 0% | 80% |
| | isoleucine, lysine, valine, | | |
| | Phenylalanine, inosine | | |
| 18 | Cysteine, Cystine, Glutamine, Proline, | 0% | 90 95% |
| | isoleucine, lysine, valine, Phenylalanine | | |

### Example 3—Assessment of a Combination of L-isoleucine and L-valine with STRAIN-1

Based on the results in Example 2, a third set of tests was conducted to evaluate activation of a combination of L-isoleucine and L-valine, without any other amino acids, in comparison to the L-alanine Control Composition and the Buffer Control Composition. The L-isoleucine and L-valine composition (“I-V Composition”) used was a nutrient-germinant composition in accordance with a preferred example of the disclosure and comprised 1 ml/L Kathon, 45 g/L Potassium Chloride, 10 g/L Monosodium Phosphate, 15 g/L Disodium Phosphate, 40 g/L L-isoleucine, and 85 g/L L-valine.

These tests were also completed with STRAIN-1. These tests also used the same incubation temperature of 42° C., same amount of STRAIN-1 spores (1 mg), and the same sampling procedure at 1 hour and 24 hours as previously described and used in Examples 1 and 2.

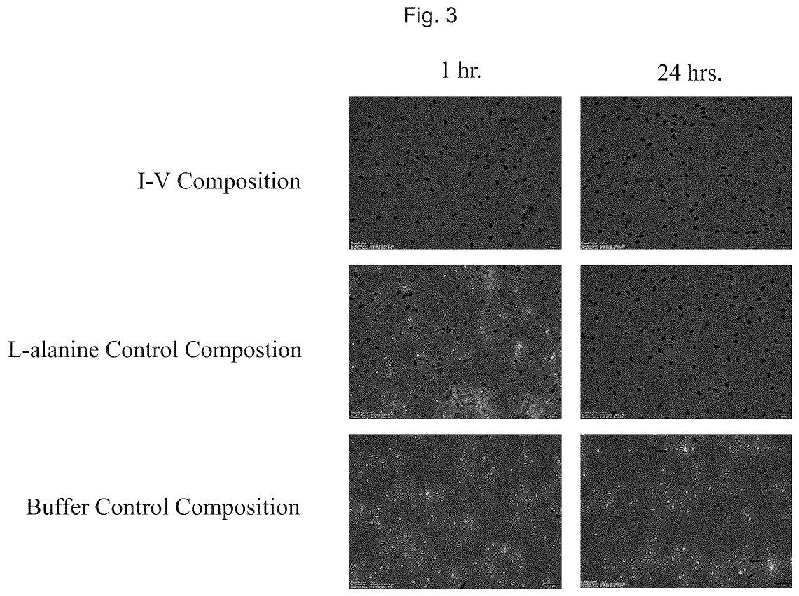

As shown in FIG. 3 (with images taken after 1 hour of incubation and after 24 hours of incubation) for each germinant solution tested, dormant spores appeared phase-bright, while activated spores appeared phase-dark. The results of these tests are shown in Table 3.

As can be seen in Table 3, the I-V Composition according to a preferred example had a 95% activation rate after only 1 hour and a 100% activation rate after 24 hours. These results are far better than the L-alanine Control Composition. These results are also surprising given (1) the zero percent activation rate after 1 hour in Table 2 for sample numbers 7, 8, 17, and 18 that included L-isoleucine and L-valine with other amino acids and (2) the zero or near zero percent activation rate after one hour for the other sample numbers in Table 2 that included L-isoleucine or L-valine with other amino-acids.

TABLE 3
Validation of the synergistic effects of L-isoleucine and L-valine

| | | STRAIN-1 | |
|---|---|---|---|
| | | Activation | Activation |
| Sample | Germinant | Rate (%) at | Rate (%) |
| No. | Composition | 1 hr. | at 24 hrs. |
| 1 | I-V Composition | 95% +/− 0 | 95% +/− 0 |
| 2 | L-alanine Control | 20% +/− 0 | 95% +/− 0 |
| | Composition | | |
| 3 | Buffer Control | 0% | 0% |
| | Composition | | |

### Example 4—Assessment of I-V Composition with Various Wild-Type Bacillus

Based on the results in Example 3, a fourth set of tests was conducted to evaluate activation of the I-V Composition in comparison to the L-alanine Control composition. These tests were completed with nine wild-type strains. The nine wild-type Bacillus species strains used were STRAIN-2, STRAIN-3, STRAIN-4, STRAIN-5, STRAIN-6, STRAIN-7, STRAIN-8, STRAIN-9, and STRAIN-10. Like STRAIN-1, none of STRAIN-2 through STRAIN-10 have been identified yet as being in a particular species and all are naturally occurring Bacillus. These tests also used the same amount of spores (1 mg) and the same sampling procedure at 1 hour and 24 hours as previously described and used in Examples 1-3. However, these tests were repeated using three different incubation temperatures, specifically 25° C., 35° C., and 42° C., to further evaluate the effects of temperature on germination. Since the optimal temperature for Bacillus growth is generally considered to be 37° C., temperatures above this are considered heat shock conditions under which better germination/activation results are expected and temperatures below this would be expected to have worse germination/activation results. The I-V Composition used was the same as that used in Example 3. The same L-alanine Control Composition was also tested, but the Buffer Control Composition was not tested.

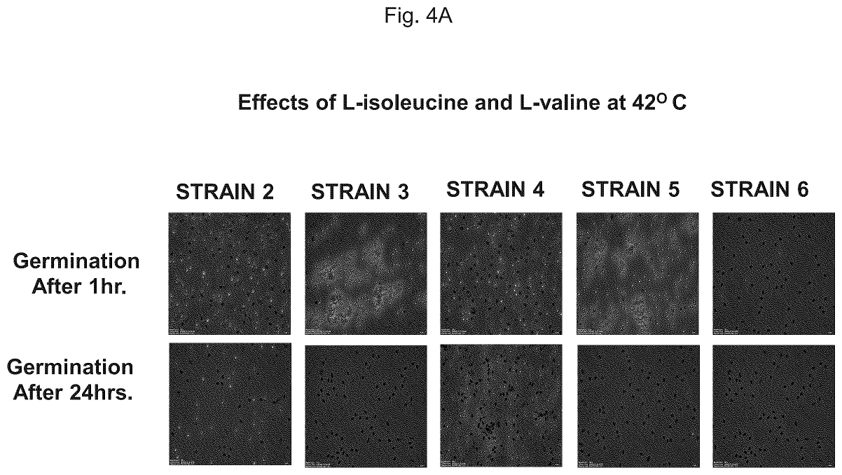

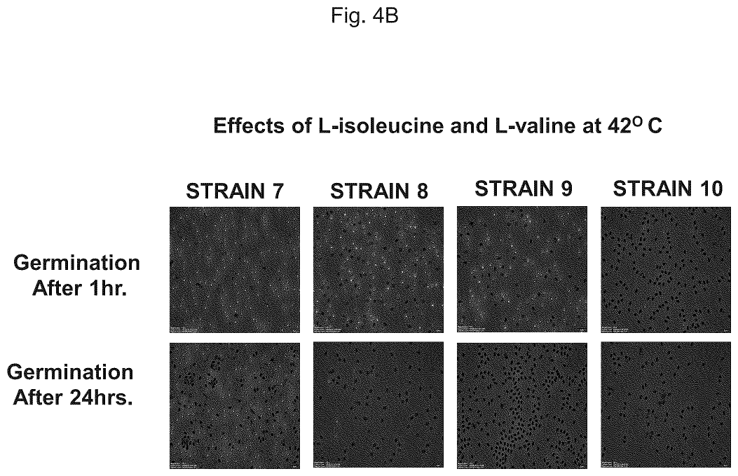

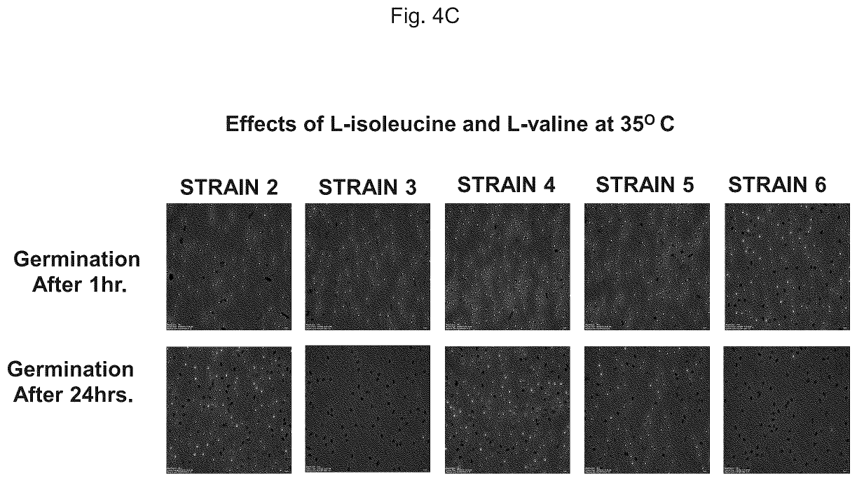

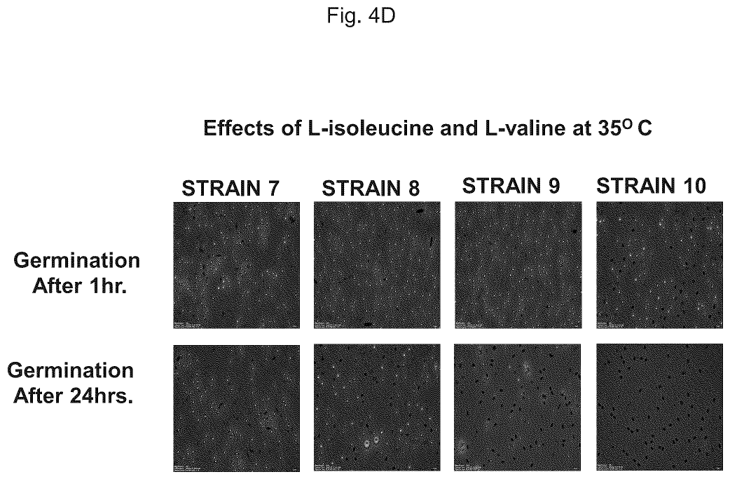

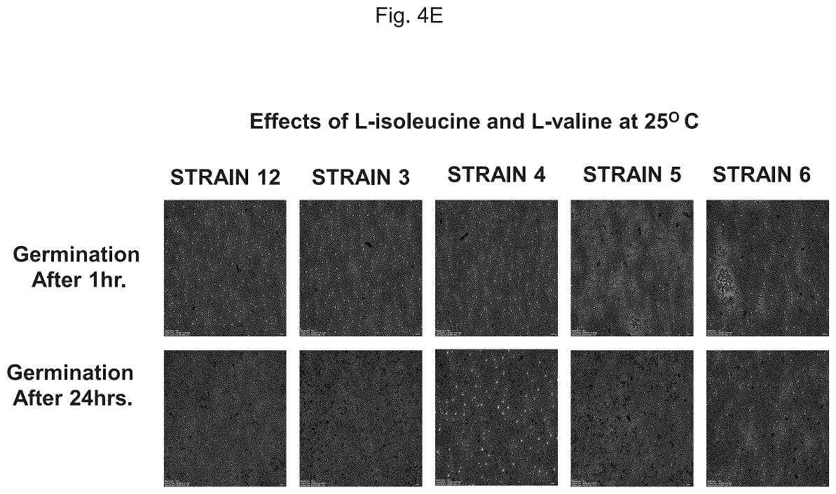

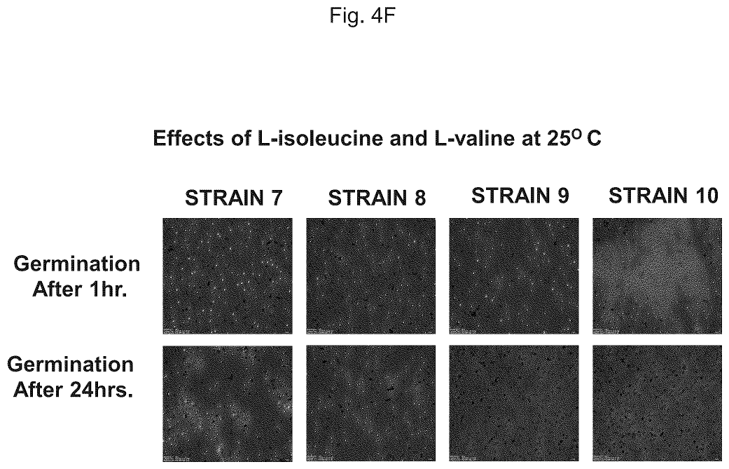

FIGS. 4A-4F show images taken after 1 hour of incubation and after 24 hours of incubation) for each Bacillus strain tested at each of the incubation temperatures, with dormant spores appearing phase-bright, while activated spores appeared phase-dark. The results at each incubation temperature are included in Tables 4A, 4B, and 4C below.

As can be seen in Tables 4A-4C, most wild strains remarkably exhibited some level of activation across all temperatures tested when using I-V Composition. However, the highest activation rate was consistently observed at 42° C. (Table 4A). One wild strain (STRAIN-7) displayed a 50% activation rate after 24 hours at 42° C. in I-V Composition and a mere 10% activation rate at 35° C. Of the remaining eight strains, four (STRAIN-2, STRAIN-4, STRAIN-5, and STRAIN-8) exhibited activation rates exceeding 30% but less than 50% at 35° C. in I-V Composition after 24 hours, while four others (STRAIN-3, STRAIN-6, STRAIN-9, STRAIN-10) displayed activation rates ranging from 77% to 95% after 24 hours (Table 4B). Only one, STRAIN-7, still had a low activation rate of 10% after 24 hours at 35° C. in I-V Composition. The lower temperature (25° C.) significantly diminished I-V Composition spore activation potency and some of the best activation rates were between 10 and 40% after 24 hours (Table 4C). In all the experiments, L-alanine Control Composition was used as control. Phase-contrast microscopy confirmed that L-alanine Control Composition treated super-dormant wild spores exhibited no discernible germination. This further proof the efficacy of I-V Composition.

More specifically, the results at 25° C. show using I-V Composition is better at activation for most of the wild-type strains than the L-alanine Control Composition. Only one strain (STRAIN-2) showed any activation with either composition after 1 hour. None of the strains showed any activation even after 24 hours with the L-alanine Control Composition, but 6 of the 9 strains showed promising results with the I-V Composition after 24 hours. The activation rates after 24 hours with the I-V Composition for these 6 strains range from 10% to 40%.

The results are significantly improved for the I-V Composition at 35° C., but still zero percent activation for all strains with the L-alanine Control Composition even after 24 hours. Two of the strains (STRAIN-6 and STRAIN-10) showed 50% activation after 1 hour in I-V Composition and four of the strains (STRAIN-3, STRAIN-6, STRAIN-9, and STRAIN-10) were well over 50% activation in I-V Composition after 24 hours. All of the strains had achieved at least 10% activation, with 8 of the strains over 30% activation, in I-V Composition after 24 hours.

Even better results are seen at 42° C. for I-V Composition. The L-alanine Control Composition still had zero percent activation at 42° C. after 24 hours for all 9 strains. In contrast, I-V Composition resulted in 5 strains (STRAIN-3, STRAIN-4, STRAIN-6, STRAIN-9, and STRAIN-10) being over 50% after 1 hour and all 9 strains were over 50% after 24 hours. A comparison of results at 42° C. and 35° C. also shows that the temperature increase to 42° C. resulted in significant increases in activation rates after 1 hour in I-V Composition for several of the strains. STRAIN-3, STRAIN-4, STRAIN-6, STRAIN-9, and STRAIN-10 each increased the activation rate by more than 40 percentage points when going from 35° C. to 42° C. Additionally, STRAIN-3 increased by more than 80 percentage points when going from 35° C. to 42° C.

TABLE 4A
Comparing the rates of activation at 42° C.

| | 42° C. | | | |
|---|---|---|---|---|
| | 1 hr. | | 24 hrs. | |
| | L-alanine | | L-alanine | |
| | Control | I-V | Control | I-V |
| | Composition | Composition | Composition | Composition |
| | Activation | Activation | Activation | Activation |
| Strains | Rate (%) | Rate (%) | Rate (%) | Rate (%) |
| STRAIN-2 | 0 | 20 +/− 0 | 0 | 60 +/− 0 |
| STRAIN-3 | 0 | 90 +/− 0 | 0 | 100 +/− 0 |
| STRAIN-4 | 0 | 53 +/− 6 | 0 | 73 +/− 6 |
| STRAIN-5 | 0 | 8 +/− 6 | 0 | 87 +/− 6 |
| STRAIN-6 | 0 | 95 +/− 0 | 0 | 100 +/− 0 |
| STRAIN-7 | 0 | 8 +/− 3 | 0 | 50 +/− 0 |
| STRAIN-8 | 0 | 20 +/− 0 | 0 | 80 +/− 10 |
| STRAIN-9 | 0 | 57 +/− 6 | 0 | 96 +/− 1 |
| STRAIN-10 | 0 | 95 +/− 0 | 0 | 99 +/− 0 |

TABLE 4B
Comparing the rates of activation at 35° C.

| | 35° C. | | | |
|---|---|---|---|---|
| | 1 hr. | | 24 hrs. | |
| | L-alanine | | L-alanine | |
| | Control | I-V | Control | I-V |
| | Composition | Composition | Composition | Composition |
| | Activation | Activation | Activation | Activation |
| Strains | Rate (%) | Rate (%) | Rate (%) | Rate (%) |
| STRAIN-2 | 0 | 5 +/− 0 | 0 | 33 +/− 6 |
| STRAIN-3 | 0 | 7 +/− 3 | 0 | 83 +/− 6 |
| STRAIN-4 | 0 | 5 +/− 0 | 0 | 33 +/− 6 |
| STRAIN-5 | 0 | 0 | 0 | 40 +/− 0 |
| STRAIN-6 | 0 | 50 +/− 0 | 0 | 92 +/− 3 |
| STRAIN-7 | 0 | 0 | 0 | 10 +/− 0 |
| STRAIN-8 | 0 | 5 +/− 0 | 0 | 37 +/− 6 |
| STRAIN-9 | 0 | 0 | 0 | 77 +/− 6 |
| STRAIN-10 | 0 | 50 +/− 0 | 0 | 95 +/− 0 |

TABLE 4C
Comparing the rates of activation at 25° C.

| | 25° C. | | | |
|---|---|---|---|---|
| | 1 hr. | | 24 hrs. | |
| | L-alanine | | L-alanine | I-V |
| | Control | I-V | Control | |
| | Composition | Composition | Composition | Composition |
| | Activation | Activation | Activation | Activation |
| Strains | Rate (%) | Rate (%) | Rate (%) | Rate (%) |
| STRAIN-2 | 0 | 3 +/− 3 | 0 | 20 +/− 0 |
| STRAIN-3 | 0 | 0 | 0 | 30 +/− 0 |
| STRAIN-4 | 0 | 0 | 0 | 0 |
| STRAIN-5 | 0 | 0 | 0 | 10 +/− 0 |
| STRAIN-6 | 0 | 0 | 0 | 20 +/− 0 |
| STRAIN-7 | 0 | 0 | 0 | 0 |
| STRAIN-8 | 0 | 0 | 0 | 0 |
| STRAIN-9 | 0 | 0 | 0 | 30 +/− 0 |
| STRAIN-10 | 0 | 0 | 0 | 40 +/− 0 |

### Example 5—Assessment of Different Concentrations of L-isoleucine and L-valine with STRAIN-1

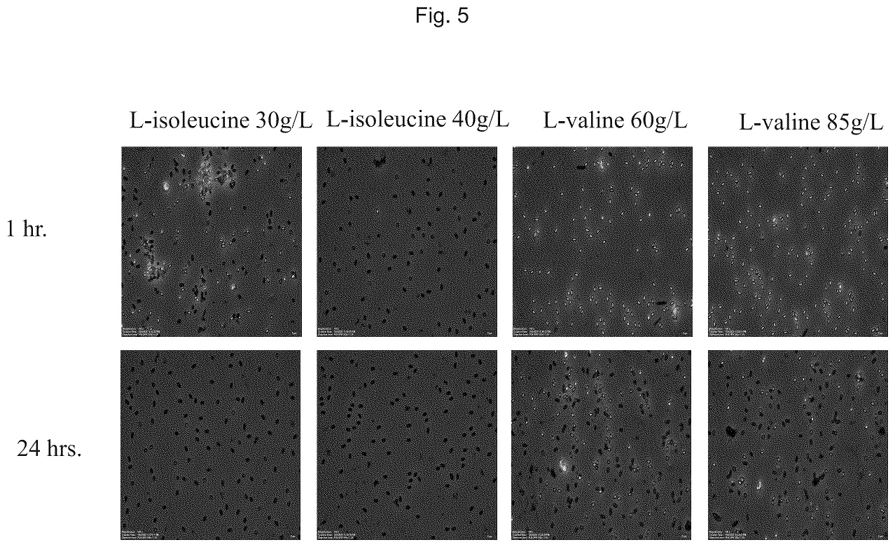

A factor that has been shown to affect rates of spore activation is the germinant concentration and studies show that a lower nutrient germinant concentration generates a low level of GR-bound germinants and as such decreases the rates of germination commitment. To study the effects of nutrient germinant concentration in inducing activation in wild-type super-dormant Bacillus strains, two different concentrations of L-isoleucine and L-valine were tested with STRAIN-1. These results are shown in Table 5. The concentration tested for L-isoleucine was 30 g/L (sample number 1) and 40 g/L (sample number 2) and for L-valine was 60 g/L (sample number 3) and 85 g/L (sample number 4). Each sample composition also had the following ingredients: 1 ml/L Kathon (an industrial preservative), 45 g/L potassium chloride, 10 g/L monosodium phosphate, and 15 g/L disodium phosphate. These tests also used the same incubation temperature of 42° C., same amount of STRAIN-1 spores (1 mg), and the same sampling procedure at 1 hour and 24 hours as previously described and used in Examples 1 and 2. FIG. 5 shows images for each sample taken after 1 hour of incubation and after 24 hours of incubation). Again, dormant spores appeared phase-bright, while activated spores appeared phase-dark. The results of these tests are shown in Table 5.

As can be seen in Table 5, a higher concentration of L-isoleucine had a higher impact on activation after one hour which is very vital in the commitment phase. Sample number 2 had a higher concentration of L-isoleucine and resulted in 25% higher activation rate at 1 hour compared to sample number 1. However, the L-valine samples did not induce any activation after 1 hour, even at the higher concentration. Sample number 4 with a higher concentration of L-valine showed 15% more activation after 24 hours than the lower concentration in sample number 3.

TABLE 5
Concentration dependent study of L-isoleucine and L-valine.

| | | STRAIN-1 | |
|---|---|---|---|
| | | Activation | Activation |
| Sample | Germinant | Rate (%) | Rate (%) |
| No. | Concentration | at 1 hr. | at 24 hrs. |
| 1 | L-isoleucine 30 g/L | 70% +/− 0 | 95% +/− 0 |
| 2 | L-isoleucine 40 g/L | 95% +/− 0 | 99% +/− 0 |
| 3 | L-valine 60 g/L | 0% | 70% +/− 0 |
| 4 | L-valine 85 g/L | 0% | 95% +/− 0 |

### Example 6—Pre-Activation with I-V Composition

Additional tests were conducted using two methods to pre-activate the spores with an I-V Composition. In a first method according to a preferred example, an I-V Composition (same as used in prior examples) was added to the fermented broth for STRAIN-1 towards the end of the fermentation process (between 30 minutes of the end and the end of the process) previously described and bioprocessed for one hour to pre-activate the spores of STRAIN-1. The broth was then spray dried at 117° C. and a first powder of pre-activated spores was harvested.

In a second method according to another preferred example, 5 g of super-dormant, fully grown, wild-type spores of STRAIN-1 were added to 250 ml of the Buffer Control Composition into a plastic bottle and mixed thoroughly. Next glass beads and 4 drops of Tween (a non-ionic surfactant) were added and subsequently shaken until well mixed. Then 100 ml of an I-V Composition (same as used in the prior examples) was added and heated in a water bath at 42° C. for 3 minutes. The solution was then transferred into a beaker and immediately cooled in an ice bucket before being spray dried. The solution was spray dried at 117° C. and a second powder of pre-activated spores was harvested.

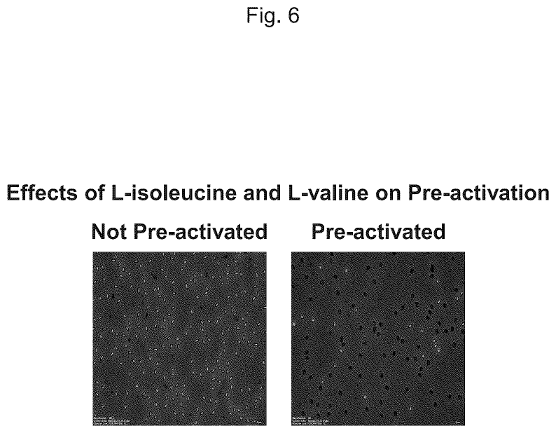

After spray drying, the first powder and the second powder had about 90% pre-activated spores. FIG. 6 shows images of a powdered nutrient-spore composition after mixing with DI water, but prior to incubation, when observed under a phase-contrast microscope. The image on the right is for a powdered nutrient-spore composition with pre-activated spores (the first powder from bioprocessing described above) and the image on the left is for a powdered nutrient-spore composition with spores that are not pre-activated. The spores in both images are STRAIN-1. Again, dormant spores appear phase-bright and pre-activated spores appear light blue (or “phase light”), not completely dark and not completely phase-bright, but in between. The phase-light specks indicate that the spores are pre-activated, meaning they have completed the “commitment” step toward activation. Additionally, when the first powder and the second powder with pre-activated spores of STRAIN-1 were each mixed in DI water and incubated at 42° C. for 1 hr, they each yielded over 95% activated spores.

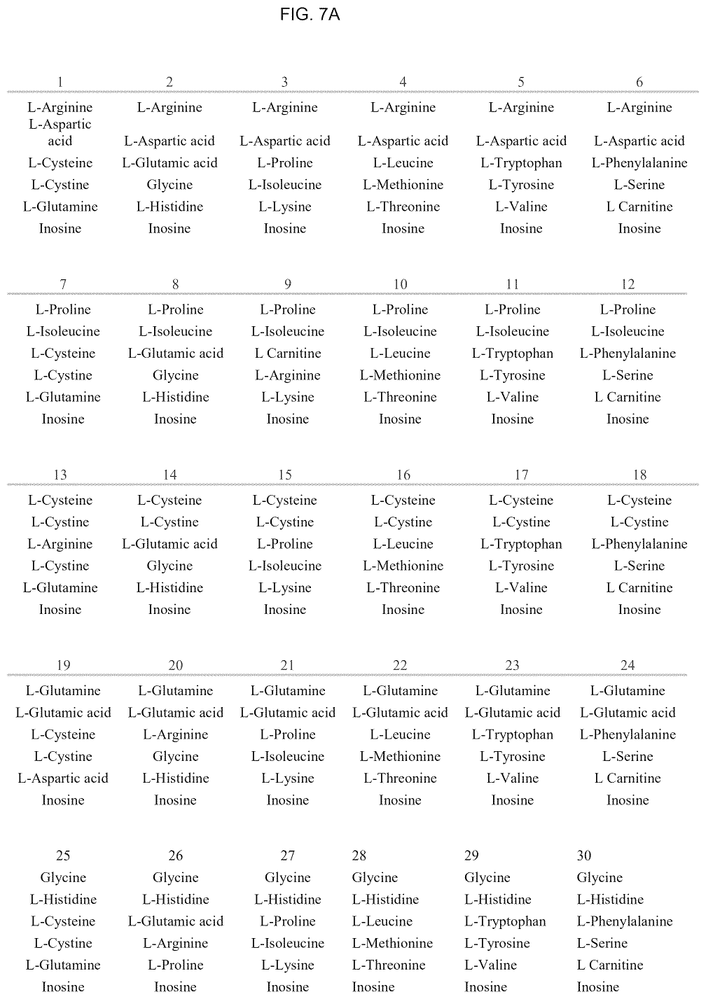

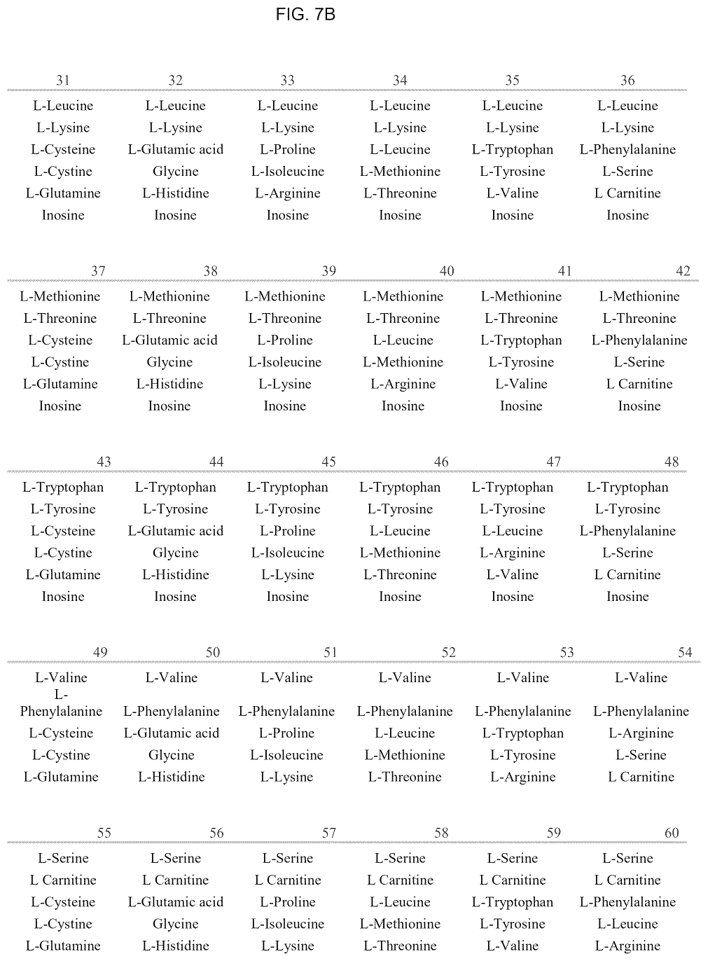

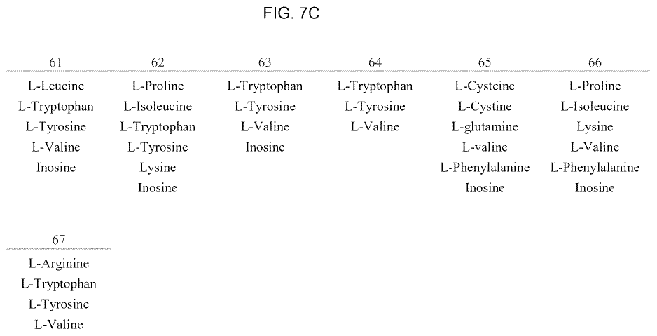

FIGS. 7A-7C contains a list of other combinations of L-amino acids that were tested using the same procedures as in Example 2. The activation rate of most these combinations after 24 hours of incubation at 42° C. were 0% and the ones that induced activation are listed in Table 2.

It will be appreciated that germinant compositions and/or methods of germinating spores, particularly wild strains and/or super-dormant strains of bacteria, and more particularly super-dormant Bacillus species, disclosed herein may include one or more of the following examples:

Example 1. A germinant composition for activating one or more bacteria spores, the germinant composition comprising: L-isoleucine or L-valine or both; and wherein the one or more bacteria spores are wild-type spores or super-dormant spores or both.

Example 2. The germinant composition of example 1 further comprising: (1) a buffer and (2) a source of potassium.

Example 3. The germinant composition of example 2 wherein the buffer comprises monosodium phosphate or disodium phosphate.

Example 4. The germinant composition of any one of examples 1 or 2 wherein the buffer comprises monosodium phosphate and disodium phosphate.

Example 5. The germinant composition of any one of examples 2 to 4 wherein the source of potassium comprises potassium chloride.

Example 6. The germinant composition of any one of examples 1 to 5 further comprising the one or more bacteria spores.

Example 7. The germinant composition of any one of examples 1 to 6 further comprising an industrial preservative.

Example 8. The germinant composition of any one of examples 1 to 7 further comprising a germination inhibitor.

Example 9. The germinant composition of any one of examples 6 to 8 wherein the one or more bacteria spores are pre-activated.

Example 10. The germinant composition of any one of examples 6 to 8 wherein the one or more bacteria spores are not pre-activated.

Example 11. The germinant composition of any one of examples 1 to 10 wherein the germinant composition does not include any other L-amino acids.

Example 12. The germinant composition of any one of examples 1 to 11 wherein the germinant composition does not include any aspartic acid or inosine.

Example 13. The germinant composition of any one of examples 7 to 12 wherein the industrial preservative comprises one or more of methyl chloro isothiazolinone, methyl isothiazolinone, propylparaben, or methylparaben.

Example 14. The germinant composition of any one of examples 7 to 13 wherein the germinant composition comprises 0.5-3.3 g/L of the industrial preservative.

Example 15. The germinant composition of any one of examples 2 to 14 wherein the germinant composition comprises 23 to 45 g/L of the source of potassium and 4 to 20 g/L of the buffer.

Example 16. The germinant composition of any one of examples 2 to 15 wherein the germinant composition comprises 9.5 to 20 g/L of the buffer and the buffer comprises 7.5 to 15 g/L of disodium phosphate and 2.5 to 5 g/L of monosodium phosphate.

Example 17. The germinant composition of any one of examples 1 to 16 further comprising one or more of L-arginine, L-tryptophan, L-tyrosine, L-phenylalanine, L-cysteine, L-cystine, L-glutamine, L-proline, or L-lysine.

Example 18. The germinant composition of any one of examples 1 to 17 further comprising aspartic acid or inosine or both.

Example 19. The germinant composition of any one of examples 1 to 16 further comprising (1) 62 to 159 g/L total of one or more of L-arginine, L-tryptophan, L-tyrosine, L-phenylalanine, L-cysteine, L-cystine, L-glutamine, L-proline, and L-lysine, (2) aspartic acid, (3) inosine, or (4) a combination thereof.

Example 20. The germinant composition of any one of examples 2 to 19 wherein the germinant composition comprises around 55 to 95% of a nutrient-germinant composition and around 5 to 13% of a spore composition; wherein the nutrient-germinant composition comprises the buffer, the source of potassium, and the L-isoleucine or L-valine or both; and wherein the spore composition comprises the one or more bacteria spores.

Example 21. The germinant composition of any one of examples 1 to 20 wherein the one or more bacteria spores comprises spores of Bacillus.

Example 22. The germinant composition of any one of examples 1 to 21 wherein the germinant composition is in a dry form.

Example 23. The germinant composition of any one of examples 1 to 21 wherein the germinant composition is in a liquid form and comprises water.

Example 24. A method of pre-activating one or more bacteria spores, the method comprising the following steps: forming a mixed liquid solution comprising a nutrient-germinant composition and the one or more bacteria spores; treating the mixed liquid solution by (1) bioprocessing the mixed liquid solution for a bioprocessing period of time or (2) heat treating the mixed liquid solution for a heat treatment period of time; and drying the mixed liquid solution after the treating step to form a dry nutrient-spore composition with the one or more bacteria spores in a pre-activated state; wherein the nutrient-germinant composition comprises the germinant composition of any one of examples 1 to 23; and wherein the one or more bacteria spores are wild-type spores or super-dormant spores or both.

Example 25. The method of example 24 wherein the drying step comprises spray drying at a first temperature in a first range of around 117 to 130° C.

Example 26. The method of any one of examples 24 to 25 wherein the treating step comprises the bioprocessing step and the bioprocessing period of time is around 10 minutes to 30 minutes.

Example 27. The method of any one of examples 24 to 25 wherein the treating step comprises the heat treating step, and wherein the heat treating step comprises heating the mixed liquid solution to a second temperature in a second temperature range of around 35 to 45° C. and the heat treatment period of time is around 180 seconds to 10 minutes.

Example 28. The method of example 25 wherein the heat treating step further comprises: mixing the one or more bacteria spores with a buffer composition in a container; adding a surface area enhancement media to the container; coating the surface area enhancement media by shaking the container or stirring; adding a surfactant to the container; cooling the mixed liquid solution in an ice bath after the heat treatment period of time; and wherein the forming step comprises adding the nutrient-germinant composition to the container.

Example 29. The method of any one of examples 24 to 28 wherein the one or more bacteria spores comprises spores of Bacillus.

Example 30. A method of activating one or more bacteria spores, the method comprising the following steps: heating a nutrient-spore solution to a temperature in a temperature range of around 26 to 60° C. for an incubation period of around 30 minutes to 36 hours to form an incubated bacteria solution; wherein the nutrient-spore solution comprises a nutrient-germinant composition and a spore composition; wherein the nutrient-germinant composition comprises L-isoleucine or L-valine or both; wherein the spore composition comprises the one or more bacteria spores; and wherein the one or more bacteria spores are wild-type spores or super-dormant spores or both.

Example 31. The method of example 30 wherein the nutrient-germinant composition further comprises (1) a buffer and (2) a source of potassium.

Example 32. The method of any one of examples 30 to 31 wherein the buffer comprises monosodium phosphate or disodium phosphate or both; and wherein the source of potassium comprises potassium chloride.

Example 33. The method of any one of examples 30 to 32 wherein the nutrient-germinant composition further comprises an industrial preservative and wherein the nutrient-germinant composition is in a liquid form.

Example 34. The method of example 33 wherein the industrial preservative comprises one or more of methyl chloro isothiazolinone, methyl isothiazolinone, propylparaben, or methylparaben.

Example 35. The method of any one of examples 30 to 34 wherein the nutrient-germinant composition further comprises a germination inhibitor.

Example 36. The method of any one of examples 30 to 35 wherein the one or more bacteria spores are pre-activated.

Example 37. The method of any one of examples 30 to 36 wherein the spore composition comprises 40% to 60% NaCl and 60% to 40% spores, by weight.

Example 38. The method of any one of examples 30 to 37 wherein the nutrient-germinant composition further comprises aspartic acid.

Example 39. The method of any one of examples 30 to 38 wherein the nutrient-germinant composition further comprises inosine.

Example 40. The method of any one of examples 30 to 39 wherein the nutrient-germinant composition further comprises 23 to 45 g/L of the potassium chloride, 4 to 20 g/L total of the buffer, 0.5-3.3 g/L of the industrial preservative, and water.

Example 41. The method of any one of examples 30 to 40 wherein the nutrient-germinant composition further comprises one or more of L-arginine, L-tryptophan, L-tyrosine, L-phenylalanine, or L-cysteine.

Example 42. The method of any one of examples 30 to 41 wherein the nutrient-germinant composition further comprises one or more of L-cystine, L-glutamine, L-proline, or L-lysine.

Example 43. The method of any one of examples 30 to 42 wherein the nutrient-germinant composition further comprises (1) 62 to 159 g/L total of one or more of L-arginine, L-tryptophan, L-tyrosine, L-phenylalanine, L-cysteine, L-cystine, L-glutamine, L-proline, and L-lysine, (2) aspartic acid, (3) inosine, or (4) a combination thereof.

Example 44. The method of any one of examples 30 to 43 wherein the nutrient-spore solution comprises around 55 to 95% of the nutrient-germinant composition and around 5 to 13% of the spore composition.

Example 45. The method of any one of examples 30 to 44 wherein the method is carried out at or near a point-of-use of the incubated bacteria solution.

Example 46. The method of any one of examples 30 to 45 further comprising: forming the nutrient-spore solution by mixing the nutrient-germinant composition and the spore composition.

Example 47. The method of any one of examples 30 to 46 wherein the nutrient-germinant composition is in a dry or powdered form and the forming step further comprises adding and mixing water with the nutrient-germinant composition and the spore composition.

Example 48. The method of any one of examples 30 to 47 wherein the nutrient-germinant composition is in a concentrated liquid form and the forming step further comprises adding and mixing water with the nutrient-germinant composition and the spore composition.

Example 49. The method of any one of examples 30 to 48 wherein the nutrient-germinant composition further comprises an industrial preservative.

Example 50. The method of any one of examples 30 to 49 wherein the nutrient-spore solution is a liquid nutrient-spore solution and the nutrient-spore solution further comprises an industrial preservative.

Example 51. The method of any one of examples 30 to 50 wherein the nutrient-spore solution is in a concentrated liquid form and the method further comprises adding and mixing water with the nutrient-spore solution prior to or during the heating step.

Example 52. The method of any one of examples 30 to 51 wherein the incubated bacteria solution comprises at least 50% activated bacteria when the incubation period is at least 24 hours.

Example 53. The method of any one of examples 30 to 51 wherein the incubated bacteria solution comprises at least 70% activated bacteria when the incubation period is at least 24 hours.

Example 54. The method of any one of examples 30 to 51 wherein the incubated bacteria solution comprises at least 90% activated bacteria when the incubation period is at least 24 hours.

Example 55. The method of any one of examples 30 to 54 wherein the incubated bacteria solution comprises at least 50% activated bacteria when the incubation period is at least 1 hour and the temperature range is 40 to 60° C.

Example 56. The method of any one of examples 30 to 55 wherein the nutrient-germinant composition does not include any other L-amino acids.

Example 57. The method of any one of examples 30 to 56 wherein the nutrient-germinant composition does not include any aspartic acid or inosine.

Example 58. The method of any one of examples 30 to 57 wherein the one or more bacteria spores are not pre-activated.

Example 59. The method of any one of examples 30 to 57 wherein the one or more bacteria spores are pre-activated according to any one of examples 24 to 29.

Example 60. The method of any one of examples 30 to 59 wherein the nutrient-germinant composition comprises the germinant composition according to any one of example 1 to 23.

Example 61. A method of activating one or more bacteria spores, the method comprising the following steps: heating a nutrient-spore solution to a temperature in a temperature range of around 26 to 60° C. for an incubation period of around 30 minutes to 36 hours to form an incubated bacteria solution, wherein the nutrient-spore solution comprises a germinant composition according to any one of examples 1 to 23.

Example 62. The method of any example 61 wherein the incubated bacteria solution comprises at least 50% activated bacteria when the incubation period is at least 24 hours.

Example 63. The method of any one of examples 61 to 62 wherein the incubated bacteria solution comprises at least 70% activated bacteria when the incubation period is at least 24 hours.

Example 64. The method of any one of examples 61 to 63 wherein the incubated bacteria solution comprises at least 90% activated bacteria when the incubation period is at least 24 hours.

Example 65. The method of any one of examples 61 to 64 wherein the incubated bacteria solution comprises at least 50% activated bacteria when the incubation period is at least 1 hour and the temperature range is 40 to 60° C.

Example 66. The method of any one of examples 61 to 65 wherein the one or more bacteria spores are wild-type spores or super-dormant spores or both.

Example 67. The method of any one of examples 61 to 66 wherein the one or more bacteria spores are Bacillus spores.

As used herein, the terms “comprises,” “comprising,” “includes,” “including,” “has,” “having,” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: (1) A is true (or present), and B is false (or not present), (2) A is false (or not present), and B is true (or present), and (3) both A and B are true (or present). Also, the use of “a” or “an” is employed to describe elements and components described herein. This is done merely for convenience and to give a general sense of the scope of the disclosure. This description should be read to include one or at least one, and the singular also includes the plural unless it is obvious that it is meant otherwise.

Any ingredient or method steps of a preferred example herein may be used with any other ingredients, features, components, or steps of other examples even if not specifically described with respect to that example, unless such combination is explicitly excluded herein. Any ingredient or amount of an ingredient, or method steps described as excluded with any particular preferred example herein may similarly be excluded with any other preferred example herein even if not specifically described with such example. Any ingredient or method step described in the prior art that is not described herein may be included in compositions and methods of the disclosure and claims or may be excluded from the disclosure and claims.

All numerical values for amounts of ingredients, ratios, temperatures, times, and other numeric values herein described as a range specifically include any individual value or ratio within such ranges and any and all subset combinations within ranges, including subsets that overlap from one preferred range to a more preferred range and even if the specific subset of the range is not specifically described herein. References to “about” or “around” with respect to numerical values generally mean+/−1 for values expressed as whole numbers without a decimal place (for example, around 42° C. means 41-43° C. and around 15% means 14-16%) and +/−0.1 for values expressed with a single or more decimal place (for example, around 9.5% means 9.4-9.6% and around 18.27 g/L means 18.17-18.37 g/L).

In the foregoing specification, compositions and methods have been described with reference to specific preferred examples. Those of ordinary skill in the art will also appreciate upon reading this specification and the description of preferred examples herein that modifications and alterations to the compositions and methods may be made within the scope of the disclosure and it is intended that the scope of the disclosure disclosed herein be limited only by the broadest interpretation of the appended claims to which the inventors are legally entitled. Accordingly, the specification and figures are to be regarded in an illustrative rather than in a restrictive sense, and all such modifications are intended to be included within the scope of the disclosure.

We claim:

1. A germinant composition for activating one or more bacteria spores, the germinant composition comprising: L-isoleucine or L-valine or both; and
wherein the one or more bacteria spores are wild-type spores or super-dormant spores or both.

2. The germinant composition of claim 1 further comprising: (1) a buffer and (2) a source of potassium.

3. The germinant composition of claim 2 wherein the buffer comprises monosodium phosphate or disodium phosphate.

4. The germinant composition of claim 2 wherein the buffer comprises monosodium phosphate and disodium phosphate.

5. The germinant composition of claim 4 wherein the source of potassium comprises potassium chloride.

6. The germinant composition of claim 5 further comprising the one or more bacteria spores.

7. The germinant composition of claim 6 further comprising an industrial preservative and wherein the germinant composition is in a liquid form.

8. The germinant composition of claim 7 further comprising a germination inhibitor.

9. The germinant composition of claim 6 wherein the one or more bacteria spores are pre-activated.

10. The germinant composition of claim 6 wherein the one or more bacteria spores are not pre-activated.

11. The germinant composition of claim 2 wherein the germinant composition does not include any other L-amino acids.

12. The germinant composition of claim 11 wherein the germinant composition does not include any aspartic acid or inosine.

13. The germinant composition of claim 2 further comprising an industrial preservative, and wherein the germinant composition is in a liquid form.

14. The germinant composition of claim 13 wherein the industrial preservative comprises one or more of methyl chloro isothiazolinone, methyl isothiazolinone, propylparaben, or methylparaben.

15. The germinant composition of claim 14 wherein the germinant composition comprises 23 to 45 g/L of the source of potassium, 4 to 20 g/L of the buffer, 0.5-3.3 g/L of the industrial preservative, and water.

16. The germinant composition of claim 15 wherein the germinant composition comprises 9.5 to 20 g/L of the buffer and the buffer comprises 7.5 to 15 g/L of disodium phosphate and 2.5 to 5 g/L of monosodium phosphate.

17. The germinant composition of claim 1 further comprising one or more of L-arginine, L-tryptophan, L-tyrosine, L-phenylalanine, L-cysteine, L-cystine, L-glutamine, L-proline, or L-lysine.

18. The germinant composition of claim 17 further comprising aspartic acid or inosine or both.

19. The germinant composition of claim 1 further comprising (1) 62 to 159 g/L total of one or more of L-arginine, L-tryptophan, L-tyrosine, L-phenylalanine, L-cysteine, L-cystine, L-glutamine, L-proline, and L-lysine, (2) aspartic acid, (3) inosine, or (4) a combination thereof.

20. The germinant composition of claim 2 wherein the germinant composition comprises around 55 to 95% of a nutrient-germinant composition and around 5 to 13% of a spore composition;
wherein the nutrient-germinant composition comprises the buffer, the source of potassium, and the L-isoleucine or L-valine or both; and
wherein the spore composition comprises the one or more bacteria spores.

21. The germinant composition of claim 20 wherein the one or more bacteria spores comprises spores of Bacillus.

22. The germinant composition of claim 21 wherein the germinant composition is in a dry form.

23. The germinant composition of claim 1 wherein the one or more bacteria spores comprises spores of Bacillus.

24. A method of pre-activating one or more bacteria spores, the method comprising the following steps:
forming a mixed liquid solution comprising a nutrient-germinant composition and the one or more bacteria spores;
treating the mixed liquid solution by (1) bioprocessing the mixed liquid solution for a bioprocessing period of time or (2) heat treating the mixed liquid solution for a heat treatment period of time; and
drying the mixed liquid solution after the treating step to form a dry nutrient-spore composition with the one or more bacteria spores in a pre-activated state;
wherein the nutrient-germinant composition comprises L-isoleucine or L-valine or both; and
wherein the one or more bacteria spores are wild-type spores or super-dormant spores or both.

25. The method of claim 24 wherein the drying step comprises spray drying at a first temperature in a first range of around 117 to 130° C.

26. The method of claim 25 wherein the treating step comprises the bioprocessing step and the bioprocessing period of time is around 10 minutes to 30 minutes.

27. The method of claim 25 wherein the treating step comprises the heat treating step, and wherein the heat treating step comprises heating the mixed liquid solution to a second temperature in a second temperature range of around 35 to 45° C. and the heat treatment period of time is around 180 seconds to 10 minutes.

28. The method of claim 25 wherein the heat treating step further comprises:
mixing the one or more bacteria spores with a buffer composition in a container;
adding a surface area enhancement media to the container;
coating the surface area enhancement media by shaking the container or stirring;
adding a surfactant to the container;
cooling the mixed liquid solution in an ice bath after the heat treatment period of time; and
wherein the forming step comprises adding the nutrient-germinant composition to the container.

29. The method of claim 25 wherein the one or more bacteria spores comprises spores of Bacillus.

30. A method of activating one or more bacteria spores, the method comprising the following steps:
heating a nutrient-spore solution to a temperature in a temperature range of around 26 to 60° C. for an incubation period of around 30 minutes to 36 hours to form an incubated bacteria solution;
wherein the nutrient-spore solution comprises a nutrient-germinant composition and a spore composition;
wherein the nutrient-germinant composition comprises L-isoleucine or L-valine or both;
wherein the spore composition comprises the one or more bacteria spores; and
wherein the one or more bacteria spores are wild-type spores or super-dormant spores or both.

31. The method of claim 30 wherein the nutrient-germinant composition further comprises (1) a buffer and (2) a source of potassium.

32. The method of claim 31 wherein the buffer comprises monosodium phosphate or disodium phosphate or both; and
wherein the source of potassium comprises potassium chloride.

33. The method of claim 32 wherein the nutrient-germinant composition further comprises an industrial preservative and wherein the nutrient-germinant composition is in a liquid form.

34. The method of claim 33 wherein the industrial preservative comprises one or more of methyl chloro isothiazolinone, methyl isothiazolinone, propylparaben, or methylparaben.

35. The method of claim 33 wherein the nutrient-germinant composition further comprises a germination inhibitor.

36. The method of claim 33 wherein the one or more bacteria spores are pre-activated.

37. The method of claim 33 wherein the one or more bacteria spores are not pre-activated.

38. The method of claim 32 wherein the nutrient-germinant composition does not include any other L-amino acids.

39. The method of claim 32 wherein the nutrient-germinant composition does not include any aspartic acid or inosine.

40. The method of claim 33 wherein the nutrient-germinant composition further comprises 23 to 45 g/L of the potassium chloride, 4 to 20 g/L total of the buffer, 0.5-3.3 g/L of the industrial preservative, and water.

41. The method of claim 30 wherein the nutrient-germinant composition further comprises one or more of L-arginine, L-tryptophan, L-tyrosine, L-phenylalanine, L-cysteine, L-cystine, L-glutamine, L-proline, or L-lysine.

42. The method of claim 41 wherein the nutrient-germinant composition further comprises aspartic acid or inosine or both.

43. The method of claim 30 wherein the nutrient-germinant composition further comprises (1) 62 to 159 g/L total of one or more of L-arginine, L-tryptophan, L-tyrosine, L-phenylalanine, L-cysteine, L-cystine, L-glutamine, L-proline, and L-lysine, (2) aspartic acid, (3) inosine, or (4) a combination thereof.

44. The method of claim 30 wherein the nutrient-spore solution comprises around 55 to 95% of the nutrient-germinant composition and around 5 to 13% of the spore composition.

45. The method of claim 30 wherein the method is carried out at or near a point-of-use of the incubated bacteria solution.

46. The method of claim 45 further comprising:
forming the nutrient-spore solution by mixing the nutrient-germinant composition and the spore composition.

47. The method of claim 45 wherein the nutrient-germinant composition is in a dry or powdered form and the forming step further comprises adding and mixing water with the nutrient-germinant composition and the spore composition.

48. The method of claim 45 wherein the nutrient-germinant composition is in a concentrated liquid form and the forming step further comprises adding and mixing water with the nutrient-germinant composition and the spore composition.

49. The method of claim 45 wherein the nutrient-germinant composition further comprises an industrial preservative.

50. The method of claim 30 wherein the nutrient-spore solution is a liquid nutrient-spore solution and the nutrient-spore solution further comprises an industrial preservative.

51. The method of claim 50 wherein the nutrient-spore solution is in a concentrated liquid form and the method further comprises adding and mixing water with the nutrient-spore solution prior to or during the heating step.

52. The method of claim 30 wherein the incubated bacteria solution comprises at least 50% activated bacteria when the incubation period is at least 24 hours.

53. The method of claim 30 wherein the incubated bacteria solution comprises at least 70% activated bacteria when the incubation period is at least 24 hours.

54. The method of claim 30 wherein the incubated bacteria solution comprises at least 90% activated bacteria when the incubation period is at least 24 hours.

55. The method of claim 30 wherein the incubated bacteria solution comprises at least 50% activated bacteria when the incubation period is at least 1 hour and the temperature range is 40 to 60° C.
